(19) United States
(12) Patent Application Publication
(10) Pub. No.: US 2018/0306027 A1
(43) Pub. Date: Oct. 25, 2018

(54) Method of Assuring Dissolution of Degradable Tools

(71) Applicant: Terves Inc., Euclid, OH (US)

(72) Inventors: Andrew Sherman, Mentor, OH (US); Brian Doud, Euclid, OH (US)

(21) Appl. No.: 16/001,491

(22) Filed: Jun. 6, 2018

Related U.S. Application Data
(63) Continuation-in-part of application No. 15/701,701, filed on Sep. 12, 2017.
(60) Provisional application No. 62/599,183, filed on Dec. 15, 2017.
(60) Provisional application No. 62/398,867, filed on Sep. 23, 2016.

(51) Int. Cl. E21B 47/10; E21B 29/00; G01N 33/24

(52) U.S. Cl. CPC E21B 47/1015; E21B 33/12; G01N 33/24; E21B 29/00

(57) ABSTRACT

The use of degradable components has become a more common practice in subterranean operations for such applications as temporarily isolating zones or diverting flow. A major concern of operators in using degradable tools is the ability to ensure that the tool has completely degraded and is no longer blocking or obstructing flow. This issue can be resolved through the use of degradable components that include one or more chemical additives that are released upon the partial or full dissolution of the degradable component, and which can be used to facilitate in the dissolution of the degradable component. The degradable component can optionally include tracer elements that are released upon the partial or full dissolution of the degradable component, and which can be detected at the surface to ensure the desired degradation or removal of the degradable component as well as hydraulic access to that stage.

The present invention claims priority on U.S. Provisional Patent Application No. 62/599,183 filed Dec. 15, 2017, which is incorporated herein by reference.

The present invention is a continuation-in-part of U.S. patent application Ser. No. 15/701,701 filed Sep. 12, 2017, which in turn claims priority to U.S. Provisional Application No. 62/398,867 filed Sep. 23, 2016, which are incorporated herein by reference.

The present invention relates to the enhanced use of degradable or dissolving tools and devices used in subterranean operations such as drilling, completion, and stimulation operations used in enhanced geothermal, oil and gas, and waste disposal (injection) operations. In particular, the invention relates to degradable components that include one or more materials to control local salinity and/or pH upon the partial or full dissolution of the degradable component, and more particularly to degradable components that include one or more materials that are time released to control local salinity and/or pH upon the partial or full dissolution of the degradable component.

### BACKGROUND OF THE INVENTION

Dissolvable and degradable materials have been developed over the last twenty (20) years for the purpose of making well completion and stimulation operations more effective and efficient. Initially, soluble salts were used for temporarily diverting flows and to control tool actuation. This technology was followed by the development and introduction of dissolvable polymers which provided structural performance, thereby enabling applications in such tools as frac balls to operate shifting tools and isolate zones. More recently, dissolvable metals, including high-strength magnesium and aluminum alloys, have been developed to enable the production of complete packer and plug fabrication. Pumpable versions of these dissolvable metals have been developed (e.g., flakes, fibers, and beads) for dispersion within the fractures outside the liner or wellbore.

Increasingly, a large number of stages are used in completing a well, and longer and higher deviation laterals are produced using directional drilling. These long laterals, deep wells, and high deviations increase costs and difficulties for intervention activities (such as drill-out or retrieval of plugs) and often exceed the distances where coiled tubing intervention services can be effectively used. Components made of degradable and/or dissolvable materials are increasingly being accepted in these applications.

One of the difficulties in using components formed of degradable and/or dissolvable materials is that sometimes such components have been known to not degrade or not properly degrade. Most of these components formed of degradable and/or dissolvable materials require the presence of brine to cause the degrading and/or dissolving of the component. If gas pockets, tar, or other contaminants block access to the components formed of degradable and/or dissolvable materials, or if the salt content, temperature, or conditions of the brine are wrong for the proper degrading and/or dissolving of the component, the component can remain in the well and flow communication in the well can be reduced or prevented. Build-up of insoluble hydroxide byproduct can hinder dissolution or interact with the formation locally. Changes in pH, such as a pH increase due to the production of hydroxides, can affect the formation and inhibit the effectiveness of gel-breakers or acidizing treatments. The use of chemical pills that are pumped down into the well have been used to control pH. Also, coated solid acids have been used as gel-breakers to reduce viscosity by breaking polymer chains or hydrogels used to viscosify pumping fluids to enhance proppant transport. However, these chemical pills and gel-breakers are not effective if a degradable component has not degraded or has not properly degraded, thereby interfering with the proper flow or the flow past the degradable component.

Components formed of degradable and/or dissolvable materials can normally be easily drilled out or otherwise removed (if accessible), but at added cost, which decreases the value of using the degradable.

Although the use of tools formed of degradable and/or dissolvable materials have become a more common practice in subterranean operations for such applications as temporarily isolating zones or diverting flow, a major concern of operators in using such tools is the ability to ensure that such tool has completely degraded or dissolved and is no longer fully or partially blocking flow in a well. As such, it is therefore highly desirable if there was a method to control the local environment around the degradable tool, and/or to offset the impact of hydroxides and pH changes on the formation or downhole environment so has to ensure that the degradable and/or dissolvable materials has been properly removed from a well. It would also be desirable if there was a method in which the operator conclusively knows that the tool formed of degradable and/or dissolvable materials has been properly removed from a well.

### SUMMARY OF THE INVENTION

The present invention relates to degradable and/or dissolving tools or devices (hereinafter referred to as a “degradable component”) and the use thereof in subterranean operations such as drilling, completion, and stimulation operations used in geothermal, oil and gas, and waste disposal (injection) operations, wherein the degradable component includes one or more chemical additives (e.g., salt, buffer chemical mixture, solid acid, or other active chemical) that are released upon the partial or full dissolution of the degradable component, and which the one or more chemical additives are used to at least partially control the local chemical environment about the degradable component to a) maintain the rate of degradation of the degradable component, b) enhance or accelerate the degradation of the degradable component, c) delay or slow the rate of degradation of the degradable component, and/or d) offset, neutralize or remove the byproducts of the degradation of the degradable component. Non-limiting examples of the types of tools used in geothermal, oil and gas, and waste disposal (injection) operations that can be formed of or incorporate a degradable component are disclosed in U.S. Pat. Nos. 8,905,147; 8,717,268; 8,663,401; 8,631,876; 8,573,295; 8,528,633; 8,485,265; 8,403,037; 8,413,727; 8,211,331; 7,647,964; US Publication Nos. 2015/0239796; 2015/0299838; 2015/0240337; 2016/0137912; 2013/0199800; 2013/0032357; 2013/0029886; 2007/0181224; and WO 2013/122712; which are all incorporated herein by reference. The use of degradable components has become a more common practice in subterranean operations for such applications as temporarily isolating zones or diverting flow. A major concern of operators using degradable components is the ability to ensure that the degradable component has sufficiently or completely degraded; this concern can be addressed by ensuring that the environment about the degradable components is proper for the full or partial dissolution of the degradable component. Such proper environment can be fully or partially achieved by the inclusion of one or more chemical additives that are 1) coated on the degradable component, 2) incorporated in the composition of the degradable component, and/or 3) contained in one or more cavities of the degradable component.

In one non-limiting aspect of the present invention, one or more chemical additives are released from the degradable component as ions/atoms, molecules, and/or particles species. The one or more chemical additives generally are or include salts, acids, and/or buffer materials such as, but not limited to, alkali or alkaline metals, bicarbonates, surfactants, etc. For example, the one or more chemical additives can be an enteric-coated solid acid or buffer particle that is used to neutralize a high pH solution, thereby releasing acid only when the pH increases beyond a certain level. Such chemical additives can be used to limit or prevent Mg(OH)₂ build-up and to maintain degradation rates of the degradable component if poor fluid circulation occurs about the degradable component.

In another and/or alternative non-limiting aspect of the present invention, the degradable component can include a single chemical additive, or two or more different chemical additives. When the degradable component includes two or more different chemical additives, 1) the concentration of the two or more chemical additive can be the same or different, 2) the location of the two or more chemical additives on the degradable component can be the same or different, 3) the time of release of the two or more chemical additives from the degradable component can be the same or different, and/or 4) the rate of release of the two or more chemical additives from the degradable component can be the same or different. The one or more chemical additives can optionally have controlled release properties by one or more mechanisms such as 1) a degradable or dissolvable coating about the outer surface of the chemical additive, 2) the particle size of the chemical additive, and/or 3) the shape of the chemical additive. For example, concentrated amounts of the one or more chemical additives can be released over a short period after exposure to the targeted depth/distance in the well and/or exposure to certain pressures, temperatures and/or chemical environment in the well. The one or more chemical additives can optionally be added in a desired amount and/or concentration into holes or other features in the degradable component, and can be optionally covered, coated, plugged or sealed in or on the degradable component by a coating, a seal, or and/or adhesive. Such optional covering, coating, plug or seal can be used to 1) control the timing of release of the one or more chemical additives from the degradable component and/or 2) limit or prevent removal of the one or more chemical additives from the degradable component during handling, shipment, and placement of the degradable component in the wellbore.

In another and/or alternative non-limiting aspect of the present invention, the one or more chemical additives can be 1) incorporated uniformly throughout the degradable component, 2) added to specific locations on the surface of the degradable component surface, 3) coated on the complete surface of the degradable component, 4) placed at one or more different depths within the degradable component, 5) placed in one or more internal regions within the degradable component, and/or 6) positioned in one or more cavities of the degradable component. In one non-limiting embodiment, the one or more chemical additives are positioned in one or more internal cavities of the degradable component. A degradable plug or cap can optionally be used to control the time of release of the one or more chemical additives from the one or more cavities in the degradable component. As such the time and/or rate of degradation of the plug can be controlled by 1) the composition of the plug, 2) the size of the plug, and/or 3) the shape of the plug. Generally, the degradable plug or cap is formed of a different material from the degradable component; however, this is not required.

In another and/or alternative non-limiting aspect of the present invention, there is provided a method of influencing degradation of a degradable component comprising a) providing a degradable component (e.g., tool, device, ball, frac ball, valve, plug, etc.) that is at least partially formed of a degradable material; b) providing one or more chemical additives that are i) coated on the degradable component, ii) incorporated in the composition of the degradable component, and/or iii) contained in one or more cavities of the degradable component, said one or more chemical additives selected to influence degradation of said degradable component while said degradable component is in a wellbore; c) placing said degradable component in the wellbore; d) providing a wellbore fluid in a region about said degradable component, said wellbore fluid contacting said degradable component while said degradable component is in the wellbore; and e) at least partially releasing the one or more chemical additives from the degradable component while the degradable component is in the wellbore to affect the salinity, pH, viscosity and/or some other fluid property of the wellbore fluid that is in contact with the degradable component to thereby influence degradation of said degradable component while said degradable component is in a wellbore.

In another and/or alternative non-limiting aspect of the present invention, the one or more chemical additives can be optionally released in a controlled manner from the degradable component into the local wellbore fluid environment.

In another and/or alternative non-limiting aspect of the present invention, the one or more chemical additives can include one or more components selected from an acid, buffer compound, salt, oxidizer, water-rechemical additive, surfactant, and/or absorbent material. Non-limiting examples of the one or more chemical additives include a salt (e.g., KCl, NaCl, CaCl₂, NaBr, KBr, MgCl₂, AlCl₃, AlBr₃, BF₃, AlF₃, KI, NaI, ZnCl₂, ZnBr₂, CuCl₃, etc.), acid (e.g., carboxylic acids (steric acid, benzoic acid, maleic acid, malonic acid, etc.), solid acid (e.g., phosphoric acid, etc.), acid chloride (e.g., ethonyl chloride, benzoic chloride, etc.), and/or buffering acid (e.g., oxalic acid etc.)), sulfates (e.g., sodium sulfate, sulfur oxide, sodium bisulfate, etc.), chlorine compounds (e.g., perchlorates, etc.), acid oxidizer, and/or basic oxidizer. In one non-limiting embodiment, the one or more chemical additives constitute about 0.1-30 wt. % of the degradable component (and all values and ranges therebetween), typically the one or more chemical additives constitute about 1-30 wt. % of the degradable component, and more typically the one or more chemical additives constitute about 3-10 wt. % of the degradable component. In another and/or alternative non-limiting embodiment, the one or more chemical additives are formulated to partially or fully neutralize the formation of hydroxides in the wellbore fluid that is located about the degradable compound and/or to maintain a pH of the wellbore fluid below about 10. In one non-limiting specific configuration, the one or more chemical additives are formulated to maintain a pH of the wellbore fluid below about 8 that is located about the degradable compound, typically a pH of the wellbore fluid below 7 that is located about the degradable compound, and more typically a pH of the wellbore fluid below about 6 that is located about the degradable compound. In another and/or alternative non-limiting embodiment, the one or more chemical additives are formulated to produce about 1000-10000 ppm of chloride content (and all values and ranges therebetween) in the wellbore fluid that is located about the degradable compound, and typically about 3000-5000 ppm chloride content in the wellbore fluid that is located about the degradable compound.

In another and/or alternative non-limiting aspect of the present invention, the one or more chemical additives can optionally be in the form of particles with a particle size distribution, where the solubility or reaction rate can be optionally controlled by the particle size and/or particle size distribution. The one or more chemical additives can optionally be 1) coated with a degradable or dissolvable material and/or 2) a degradable or dissolvable matrix material can be incorporated with the one or more chemical additives to control the interaction with and/or release of the one or more chemical additives into the wellbore fluid. When the one or more chemical additives are incorporated in a degradable or dissolvable matrix material, the matrix material can optionally be selected from a water-soluble or water-reactive polymer or compound (e.g., hydrogel, absorbent material, etc.). Non-limiting examples of such water-soluble or water-reactive polymers or compounds that can be used to for the matrix material include PVA, PGA, PEG, sugar, cellulose, a poly(α-hydroxyacid) (e.g., poly(lactic acid), poly(glycolic acid), or blends thereof), poly(orthoester), poly(anhydride), poly(hydroxyl alkanoate), gelatin, chitosan, arabinogalactan, collagen, alginate, hyaluronic acid, fibrin, cellulose, and/or cellulose ether.

In another and/or alternative non-limiting aspect of the present invention, the one or more chemical additives can optionally be in the form of granules, pellets, or powders. In one non-limiting embodiment, the one or more chemical additives are in the form of a particle, which particle is a microparticle or a nanoparticle; however, this is not required. In another non-limiting embodiment, the one or more chemical additives in the form of a plurality of particles are optionally compressed to form a solid pellet. The solid pellet can include one or more different types of chemical additives or be formed of a single type of chemical additives. Generally, the size of the pellet can pass through Mesh size No. 4 to No. 140 (and all sizes therebetween).

In another and/or alternative non-limiting aspect of the present invention, the one or more chemical additives can optionally be incorporated in, or be in the form of gel, bulk scaffold, thin film or pellet.

In another and/or alternative non-limiting aspect of the present invention, the one or more chemical additives can optionally include or be used with an amphoteric species which is formulated to a) alter the rate of dissolution of the gel, bulk scaffold, thin film, or the pellet, and/or b) alter the release of the one or more chemical additives from the gel, bulk scaffold, thin film, or the pellet, thereby altering the release of the one or more chemical additives from the gel, bulk scaffold, thin film, or the pellet. In one non-limiting embodiment, the amphoteric species can serve as a diffusion barrier to the one or more chemical additives in the gel, bulk scaffold, thin film, or the pellet.

In another and/or alternative non-limiting aspect of the present invention, the one or more chemical additives can optionally be bound to the degradable component by a binder. In one non-limiting embodiment, the binder (when used) can optionally be a water-soluble cellulose ether. Non-limiting examples of water-soluble cellulose ether include, but are not limited to, methylcellulose (e.g., Methocel® A, previously designated as Methocel® MC, from The Dow Chemical Co., U.S.A. and Metalose® SM from Shin-Etsu, Ltd., Japan has a methoxyl content of 27.5-31.5 wt. % and is available in various viscosity grades, etc.), hydroxypropylmethylcellulose (e.g., Methocel® E, F, J and K, all previously designated as versions of Methocel® HG, from The Dow Chemical Co., U.S.A., and Metalose SH from Shin-Etsu, Ltd., Japan, each of which has a different chemical composition with a methoxyl content within the range of 16.5-30 wt. %, a hydroxypropoxyl content within the range of 4-32 wt. % and each of which is available in various viscosity grades, etc.). The water-soluble cellulose ether can optionally be compounded with the one or more chemical additives and a surfactant. Anionic surfactants which can be optionally used include alkali metal sulfates of linear and branched alcohols, ethoxylated alcohols, ethoxylated alkylphenols, ethoxylated acids, ethoxylated amides, oils, fatty esters, alkali metal salts of sulfonates of naphthalene, alkylnaphthalenes, naphthalene condensates, alkyl-substituted benzenes, diphenyl derivatives, α-olefins, petroleum, oils, fatty acids, as well as the alkali metal salts of dialkyl sulfosuccinates. Representative anionic surfactants that can be used include sodium or potassium dodecyl sulfate, sodium octadecyl sulfate, sodium sulfated castor oil, sodium dodecylbenzene sulfonate, sodium linear alkylate sulfonate, sodium sulfonated mineral oil, sodium petroleum sulfonate, sodium salt of naphthalenesulfonic acid-formaldehyde condensate and dioctyl sodium sulfosuccinate. The weight ratio of surfactant to cellulose ether is generally 0.005-3:1 (and all values and ranges therebetween). The binder can form 5-95 wt. % (and all values and ranges therebetween) of the mixture of binder and chemical additive. The cellulose ethers can optionally be used with or without prior humidification or similar treatment when mixed with the surfactant and the chemical additive.

In another and/or alternative non-limiting aspect of the present invention, the one or more chemical additives can optionally be added to a cavity in degradable component.

In another and/or alternative non-limiting aspect of the present invention, the one or more chemical additives can optionally be added to the outer surface of the degradable component.

In another and/or alternative non-limiting aspect of the present invention, the degradable component can include a ball, frac ball, or component in a tool such as a bridge or frac plug. Non-limiting examples of such a component are a mandrel, cone, element, or shoe.

In another and/or alternative non-limiting aspect of the present invention, the one or more chemical additives can optionally be positioned on the degradable component such that the one or more chemical additives are exposed to the wellbore fluid by mechanical action such as shear, sliding, pressure pulse, etc., and/or are exposed to the wellbore fluid by dissolution of a coating or plug covering a cavity in the degradable component, and wherein such plug or coating is the same or different material as the degradable component.

In another and/or alternative non-limiting aspect of the present invention, the one or more chemical additives can optionally be included as an additional component to the degradable component such as an extension to the shoe, a mandrel extension, lining, or cylinder, etc. The additional component that includes the one or more chemical additives can be mechanically and/or adhesively attached to one or more surfaces of the degradable component.

In another and/or alternative non-limiting aspect of the present invention, the one or more chemical additives can optionally be added as a coating or a lining to some or all of the surface and/or some other region of the degradable component.

In another and/or alternative non-limiting aspect of the present invention, the one or more chemical additives can optionally be protected from damage by a protective coating or covering as the degradable component is inserted into a wellbore and/or a protective coating or covering can be used to control wellbore fluid access to the one or more chemical additives on and/or in the degradable component.

In another and/or alternative non-limiting aspect of the present invention, the one or more chemical additives can be added to the degradable component while the one or more chemical additives are in a molten state. In one non-limiting embodiment, the one or more chemical additives are or include a molten salt or acid that is added into one or more cavities in a degradable component. In another non-limiting embodiment, the one or more chemical additives are heated to a molten state and then poured into a mold. In the mold, the one or more chemical additives can be optionally coated with a degradable coating (e.g., PVA, PGA, PLA, PEG, cellulose, or other degradable polymer). The one or more chemical additives that are coated with the degradable coating can be placed on the surface of a degradable component (e.g., plug, mandrel, shoe, barrier, disc, dart, or other component or device) and/or placed on one or more cavities in a degradable component. The molten one or more chemical additives can also be placed into or cast directly into the degradable component.

In another and/or alternative non-limiting aspect of the present invention, the one or more chemical additives are a solid acid, such as FeCl₃, AlCl₃, or Na₂SO₄. In one non-limiting embodiment, the ratio of the solid acid to the degradable metal is selected such as to shift the degradation byproducts and/or solution pH away from insoluble hydroxides to soluble sulfates or chorides or oxychlorides. As such, the one or more chemical additives are used to lower the pH of the liquid about the degradable component. Generally, the pH is lowered to less than 10, typically less than 8, and more typically less than 7.

In another and/or alternative non-limiting aspect of the present invention, the amount of the one or more chemical additives that is included on or in the degradable component is selected to ensure at least 35% solubilization (reaction) of the degradable material that partially or fully forms the degradable component. As such, the amount of the one or more chemical additives that is included on or in the degradable component is at least 35% the stoichiometric amount of the chemical additive required to cause at least 30-35% of the stoichiometric amount of the degradable material in the degradable component to solubilize or dissolve. The use of the one or more chemical additives in such amounts is used to inhibit or prevent the possibility of plugging and/or cementing of sand grains that would require subsequent intervention. In one non-limiting embodiment, the amount of the one or more chemical additives that is included on or in the degradable component is about 35-150% (and all values and ranges therebetween) the stoichiometric amount of the chemical additive required to cause 30%400% of the stoichiometric amount of the degradable material in the degradable component to solubilize or dissolve. In another non-limiting embodiment, the amount of the one or more chemical additives that is included on or in the degradable component is about 50-150% (and all values and ranges therebetween) the stoichiometric amount of the chemical additive required to cause 40-100% of the stoichiometric amount of the degradable material in the degradable component to solubilize or dissolve. In another non-limiting embodiment, the amount of the one or more chemical additives that is included on or in the degradable component is about 80-120% (and all values and ranges therebetween) the stoichiometric amount of the chemical additive required to cause 70-100% of the stoichiometric amount of the degradable material in the degradable component to solubilize or dissolve. In another non-limiting embodiment, the amount of the one or more chemical additives that is included on or in the degradable component is used to cause the a) dissolving or dissolution of the degradable material of the degradable component, and/or b) dissolving or dissolution of other degradable materials in close proximity (e.g., within 100 ft.) of the degradable component. As such, other degradable components such as valves, frac balls, liners, sleeves, CaCO₃ filter cakes, etc. that are located in close proximity to the chemical additive that is released from the degradable component can also be caused to be dissolved and/or have an increased dissolution or dissolving rate from the release of the chemical additive. As such, the release of the chemical additive can be used to facilitate in the cleanup of components in the wellbore.

Examples of stoichiometric amounts for chemical additives for magnesium are as follows:

A. 4.5 grams of FeCl₃ per gram of Mg, or 2.76 cc of FeCl₃ per cc of Mg (MgOH+⅔FeCl₃+H₂O→MgCl₂+Fe(OH)₃).

B. 3.7 grams of AlCl₃ per gram of Mg, or 1.35 cc of AlCl₃ per cc of Mg.

C. 4.94 grams of NaHSO₄ per gram of Mg, or 3.24 cc of NaHSO₄ per cc of Mg.

In another and/or alternative non-limiting aspect of the present invention, the one or more chemical additives can include two or more different salts. The use of a plurality of different salts can be used to 1) accelerate dissolution of the degradable component, 2) reduce or eliminate the sensitivity of the degradable component to wellbore fluid salinities, and 3) enable dissolvable metals to be used in freshwater wells. In one non-limiting embodiment, the chemical additive includes a mixture of salts, such as, but not limited to KCl and NaCl.

In another and/or alternative non-limiting aspect of the present invention, the one or more chemical additives can include two or more chemical additives, such as a solid acid in the interior of the degradable component or device, and a salt closer to, or on the surface of the degradable component or device to reduce or eliminate sensitivity to the chloride content present about the degradable component or device, while also creating soluble byproducts and conditions to prevent the need for subsequent intervention and wellbore cleanup.

In another and/or alternative non-limiting aspect of the present invention, the one or more chemical additives can be used for the purpose of wellbore cleanup. In one non-limiting embodiment, solid acids with controlled release timing/location can be used to remove cements, filter cakes, and deliver wellbore cleanup or gelbreaking chemistries to the wellbore. In such applications, the amount of chemical additive used is increased or maximized. For instance, a solid shape of the solid acid, salt or other active ingredient can first be formed, and then the solid shape can be 1) coated with a degradable coating, or 2) placed inside a degradable shell. The use of degradable coatings (e.g., PVA, etc.) have been proven to be particularly effective in providing a temperature-controlled release of the one or more chemical additives in a wellbore. For precise control over location of release of the one or more chemical additives, a degradable rubber wedge or wiper can be added to the degradable-encapsulated chemical additive to produce a pump-down dart, plug, or device that prevents fluid leakage around the degradable component or device until it is inserted into a desired location in the wellbore. Alternately or additionally, the degradable device can accommodate and/or be attached to a slickline or wireline so as to precisely locate its position for chemical release in the wellbore.

In another and/or alternative non-limiting aspect of the present invention, the one or more chemical additives such as a salt, solid acid, base, active chemical, or mixture (such as a eutectic salt mixture) can be melted and then poured into a cavity of the degradable component.

In another and/or alternative non-limiting aspect of the present invention, the one or more chemical additives are melted in its hydrate or water-containing form, and after it is poured into a cavity of the degradable component, the melted one or more chemical additives are continued to be heated to remove 90-100% of the water from the one or more chemical additives so that the one or more chemical additives solidify in its anhydrous, or lower H₂O content form.

In another and/or alternative non-limiting aspect of the present invention, the one or more chemical additives are melted and poured into a cavity of the degradable component so that the degradable component does not react or dissolve while the one or more chemical additives are in molten state, and which the one or more chemical additives do not cause significant degradation to the properties of the degradable component (less than 10% degradation of the hardness and/or strength of the degradable component) over a period of at least 1 month (e.g., 1-12 months, 1-6 months, 1-3, months) while the degradable component is stored in a non-liquid and dry conditions (less than 80 humidity) at ambient temperatures (e.g. 20-28° C.).

In another and/or alternative non-limiting aspect of the present invention, two or more chemical additives are mixed together to form an eutectic mixture that causes a lowering of the melting point of the mixture of the two of more chemical additives such that the melted eutectic mixture can be poured into a cavity of the degradable component without causing the degradable component to melt or otherwise be damaged or deformed.

In another and/or alternative non-limiting aspect of the present invention, the addition of the one or more chemical additives to the degradable component leads to little or no degradation to the mechanical performance of the degradable component when the degradable component is placed in compression due to the fact that the chemical additives are incompressible.

In another and/or alternative non-limiting aspect of the present invention, there is provided a degradable component such as a pill ball or other dissolvable device that has a cavity that includes a solid material formed of one or more chemical additives.

In another and/or alternative non-limiting aspect of the present invention, there is provided a degradable component such as a pill ball or other dissolvable device that has a cavity that includes a solid material formed of one or more chemical additives wherein the content of the one or more chemical additives in the degradable component are stoichiometrically equal to or greater than the amount required to fully dissolve the degradable component.

In another and/or alternative non-limiting aspect of the present invention, there is provided a degradable component such as a pill ball or other dissolvable device that has a cavity that includes a solid material formed of one or more chemical additives wherein the content of the one or more chemical additives in the degradable component are stoichiometrically equal to or greater than the amount required to fully dissolve the degradable material (e.g., metal, etc.) of the degradable component so that the one or more chemical additives can be used to facilitate in the full or partially dissolution of the degradable component and also facilitate in the dissolution of other secondary dissolvable components that are located near the dissolvable component.

In another and/or alternative non-limiting aspect of the present invention, there is provided a degradable component that includes a dissolvable metal shell encompassing a solid material formed of one or more chemical additives.

In another and/or alternative non-limiting aspect of the present invention, there is provided a degradable component that includes a dissolvable metal shell encompassing a solid material formed of one or more chemical additives, and wherein the dissolvable metal shell is used as a plug, bridge plug, or frac plug in a hydraulic fracturing operation.

In another and/or alternative non-limiting aspect of the present invention, there is provided a degradable component (e.g., ball, frac ball, valve, metal bridge plug or frac plug, stinger, pill, etc.) that contains one or more chemical additives (e.g., solid acid, salt, etc.) that enhances the rate of dissolution of the degradable component such that the degradable component is 80-100% dissolved in less than 72 hours.

In another and/or alternative non-limiting aspect of the present invention, there is provided a degradable component that is formed of a material (e.g., metal, etc.) that that can be dissolved or have its dissolving ability enhanced by one or more chemical additives, wherein the one or more chemical additives (e.g., in solid form, etc.) is sealed inside the degradable component by a water tight plug, interference fit, and/or polymer sealing compound.

In another and/or alternative non-limiting aspect of the present invention, there is provided a degradable component that is formed of a material (e.g., metal, etc.) that that can be dissolved or have its dissolving ability enhanced by one or more chemical additives, wherein the one or more chemical additives (e.g., in solid form, etc.) is sealed inside the degradable component by a water tight plug (e.g., threaded plug, etc.), and wherein the plug can have the same or different degradation rate than the degradable component.

In another and/or alternative non-limiting aspect of the present invention, there is provided a degradable component that includes a core that contains one or more chemical additives in an amount that is greater than required to fully dissolve the degradable material (e.g., metal, etc.) of the degradable component.

In another and/or alternative non-limiting aspect of the present invention, there is provided a method that includes degradable component (e.g., ball, frac ball, valve, metal bridge plug or frac plug, stinger, pill, etc.) that has a core that that contains one or more chemical additives (e.g., solid acid or other additive) in an amount that is greater than required to fully dissolve the degradable material (e.g., metal, etc.) of the degradable component, and which degradable component can withstand more than 5 ksi differential pressure on a seat or hydrostatic pressure of 5 ksi or more.

In another and/or alternative non-limiting aspect of the present invention, there is provided a method that includes a degradable component (e.g., ball, frac ball, valve, metal bridge plug or frac plug, stinger, pill, etc.) that has a core that that contains one or more chemical additives (e.g., solid acid or other additive) used to enhance the dissolving or degradation of the degradable component.

In another and/or alternative non-limiting aspect of the present invention, there is provided a method that includes a degradable component (e.g., ball, frac ball, valve, metal bridge plug or frac plug, stinger, pill, etc.) that has a core that that contains one or more chemical additives (e.g., solid acid, acidic pH buffer or other active chemical) that is contained or housed in the degradable component such that when the one or more chemical additives are released from the degradable component, the degradable component becomes more soluble in a fluid such as water that is positioned about the degradable component.

In another and/or alternative non-limiting aspect of the present invention, there is provided a degradable component that includes a degradable material such as a magnesium alloy, zinc alloy, or aluminum alloy, or other degradable metal, and the byproduct of the dissolution or corrosion of the degradable material (e.g., magnesium hydroxide, aluminum hydroxide, zinc hydroxide, or other metal hydroxide) is more soluble in the water or aqueous solution because of the presence of the one or more chemical additives (e.g., solid acid or other active chemical) in the water or fluid about the degradable component.

In another and/or alternative non-limiting aspect of the present invention, there is provided a degradable component wherein a thickness (thickness in one or more regions of the degradable component or the thickness of the shell of the degradable component) and the degradation rate of the degradable material of the degradable component is selected to control the timing of the release of the one or more chemical additives.

In another and/or alternative non-limiting aspect of the present invention, there is provided a degradable component having a cavity and a plug and wherein the shape, composition, and size of the plug is designed to control the timing of the release of the one or more chemical additives.

In another and/or alternative non-limiting aspect of the present invention, there is provided a degradable component used to deliver one or more chemical additives to a specific wellbore location in a time-controlled manner.

In another and/or alternative non-limiting aspect of the present invention, there is provided a method for delivering one or more chemical additives to a specific wellbore location in a time-controlled manner by use of a degradable component.

In another and/or alternative non-limiting aspect of the present invention, there is provided a method for delivering one or more chemical additives to a specific wellbore location in a time-controlled manner by use of a 1) degradable component that is used by a slickline or wireline, 2) a controlled orifice size in the degradable component, 3) pumping amount of fluid into the wellbore, or 4) other technique to control placement of the chemical additives delivery.

In another and/or alternative non-limiting aspect of the present invention, there is provided a degradable component that includes a chemical additive, degradable metal, or polymeric container made of one or more degradable materials with at least one area configured to control chemical exposure to the wellbore in response to time, temperature, or other response. Generally, the container is formed of 80-100% (and all values and ranges therebetween) degradable polymer and/or metal; however, this is not required. In one non-limiting configuration, the container is a cylindrically-shaped container that includes a cavity that includes one or more chemical additives. The one or more chemical additives are generally in solid form; however, this is not required. One or both ends of the container can optionally include a degradable plug to control the release of the chemical additive from the cavity of the container. Generally, the amount of chemical additive in the container is over 100% (e.g., 101-1000% and all values and ranges therebewteen, etc.) required to fully dissolve or degrade the degradable metal and/or polymer of the container; however, this is not required.

In another and/or alternative non-limiting aspect of the present invention, there is provided a degradable component that is configured to be pumped down into a wellbore, and which degradable component includes a degradable sealing ring (e.g., degradable plastic or elastomeric wiper or flap) that is used to create a seal with the wellbore.

In another and/or alternative non-limiting aspect of the present invention, there is provided a degradable component that is a pumpable component that is used to deliver a predetermined amount of one or more chemical additives to a location in the wellbore, wherein the one or more chemical additives are added at 70-130% (and all values and ranges therebewteen) of the amount calculated to solubilize the hydroxide and/or carbonate that is present in the form of a filter cake or degradable metal byproduct.

In another and/or alternative non-limiting aspect of the present invention, the degradable component can optionally include one or more tracer elements. The one or more tracer elements (e.g., tracer chemicals, chemical elements, particles, tags [RFID, microdevice, etc.], etc.) can be 1) coated on the degradable component, 2) incorporated in the composition of the degradable component, and/or 3) contained in one or more cavities of the degradable component. The one or more tracer elements that are released upon the partial or full dissolution of the degradable component can be configured to be detected at the surface of a well site or be detected at some other location so as to determine the proper removal or degradation of the degradable component. A major concern of operators using degradable components is the ability to ensure that the degradable component has sufficiently or completely degraded; this concern can be resolved through the addition of tracer elements that are released upon the partial or full dissolution of the degradable component and which tracer elements can be detected to ensure that the degradable component has been sufficiently removed. Tracer elements can be released as ions/atoms, molecules or particles species, or can be discreet devices such as RFID microchips, etc. The one or more tracer elements can be incorporated uniformly throughout the degradable component, added to specific locations on and/or in the degradable component, or placed at different depths within the degradable component. A degradable component can include a single tracer element or two or more different tracer elements. The tracer element can be 1) uniformly dispersed in the degradable component, 2) positioned in one or more regions of the degradable component, 3) coated on one or more portion of the outer surface or all of the outer surface of the degradable component, and/or 4) be concentrated in one or more regions of the degradable component.

In another and/or alternative non-limiting aspect of the present invention, the degradable component includes the addition of one or more tracer elements on an exterior and/or in an interior of the degradable component for the purpose of verifying and/or assuring that the degradable component has sufficiently degraded and/or dissolved. The tracer element is generally no more than 12700 microns in size. In one non-limiting embodiment, the tracer element is in the form of a magnetic particle, nanowire, nanocomposites, nanohorns, functionalized nanotubes, metalized nanotubes, magnetic wires, piezoelectric materials, fluorescing particle, phosphorescent compound and/or particles, compounds or molecules that can include stable isotopes, radioactive isotopes, rare earth or other specific elements that generally have an average size of less than about 10 microns in size, typically 0.001 microns to less than 10 microns (and all values and ranges therebetween), more typically less than 5 microns, still more typically less than one micron, and yet more typically less than 0.5 micron in size (e.g., nanoparticle [1-100 nm and all values and ranges therebetween]); however, this is not required. In another non-limiting embodiment, tracer elements in the form of microRFID, micro-resonant device (MRD) can have a size that is generally less than about 10000 microns and typically about 0.01 to 8000 microns (and all values and ranges therebetween). The type and/or amount of one or more tracer elements used in a particular component is non-limiting. The tracer element is selected such that it can be identified in and distinguished from the brine and/or other type of liquid exiting a well or other subterranean formation. As such, the tracer element has a different composition from the brine or other type of liquid inserted into the well or other subterranean formation, and is also different in composition from the formation composition in and about the well or other subterranean formation.

In another and/or alternative non-limiting aspect if the present invention, the degradable component can include the same or have different types of tracer elements in the degradable component. The one or more tracer elements can be 1) uniformly dispersed throughout a degradable component, 2) concentrated in one or more regions of the degradable component, and/or 3) include different types of tracer elements in different regions of the degradable component. In one one-limiting embodiment, the one or more tracer elements are incorporated in the degradable component and are designed to be released during or after the partial or full degradation of the degradable component. In another and/or non-limiting embodiment, one or more tracer elements can be placed in an internal cavity of the degradable component and a degradable or non-degradable plug or cap can be used to partially or fully close the cavity. The plug or cap can have the same or different composition as the degradable component. In another and/or alternative non-limiting embodiment, the degradable component can be configured to release a concentrated amount of tracer elements over a short period after the degradable plug or cap has been partially or fully dissolved or degraded. The one or more cavities in the degradable component can be formed by machining; however, this is not required. The one or more cavities in the degradable component can be closed by use of a plug, wherein the plug is connected to the cavity by a threaded connection, interference fit, swaged connection, etc.

In another and/or alternative non-limiting aspect if the present invention, the tracer element can be designed, after the degradable component partially or fully degrades, to release from the degradable component and be carried with fluid flow to a location at some distance from the original location where such one or more tracer elements are released from the degradable component, and which tracer elements can be detected once such tracer elements are transported to a different location from the location of the degradable component.

In another and/or alternative non-limiting aspect if the present invention, different tracer elements can be used in different regions or zones of the degradable component to provide information as to the degree to which the degradable component has degraded and/or whether a particular region of the degradable component has degraded and/or the degree to which it has been degraded. For example, a degradable component can include a certain amount of tracer element. By measuring the amount of tracer element that has been detected at the surface or at some other location, an estimation or calculation can be made regarding the degree to which the degradable component has degraded and/or the degree to which multiple degradable components have degraded. In another example, different types of tracer element can be incorporated and/or positioned at different regions of a degradable component. By measuring and/or detecting the tracer element that has been detected at the surface or at some other location, it can be determined whether a certain region of one or more degradable components have begun to degrade and to what degree that a certain region of one or more degradable components have degraded. In another and/or alternative embodiment, different tracer elements can be used in different degradable components. As such, when multiple degradable components are positioned in a well, etc., the measuring and/or detecting of a particular tracer element and/or volume of tracer element at the surface can be used to determine 1) whether a particular degradable component(s) has begun to degrade or has degraded, 2) whether a certain region of a particular degradable component(s) has begun to degrade, and/or 3) to what degree that the particular degradable component(s) or a certain region of the particular degradable component(s) has degraded.

In another and/or alternative aspect of the present invention, the tracer element can be chosen from one or more tracer elements which can include microRFID, magnetic wires, nanowires, magnetic particles, fluorescing, and phosphorescent compounds and/or particles; and/or from compounds or molecules that can include stable isotopes, radioactive isotopes, rare earth or other specific elements, as well as compounds with high sensitivity in mass spectroscopy or other analytical techniques that are sensitive to ppb levels. A variety of detectable materials can be used as the tracer element such as trackers, taggants, markers, tracking materials, and/or tracers.

In another and/or alternative aspect of the present invention, the tracer element can be a material as disclosed in U.S. Pat. No. 8,006,755 (e.g., piezoelectric materials with a perovskite crystallographic structure type such as lead zirconate titanate (PZT) and barium titanate; magnetostrictive materials such as Terfenol-D (a family of alloys of terbium, iron and dysprosium), Samfenol (a family of alloys of samarium and iron, sometimes also containing other elements such as dysprosium), and Galfenol (a family of alloys of gallium and iron, sometimes also containing other elements); U.S. Pat. No. 7,516,788 (e.g., a dye detectable by color such as “acid blue” water-soluble dyes, “oil red” oil-soluble dyes, molecular iodine, iron oxide class pigments, chrome oxide pigments, mica ferric oxide pigments, other oxide or inorganic pigments, or organic pigments; marker easily detected spectrographically such amides, amines, or phenols); U.S. Pat. No. 6,725,926 (e.g., water soluble salts such as metal salts in which the metal is selected from Groups I to VIII of the Periodic Table of the Elements as well as the lanthanide series of rare earth metals, barium, beryllium, cadmium, chromium, cesium, sodium, potassium, manganese, zinc, barium bromide, barium iodide, beryllium fluoride, beryllium bromide, beryllium chloride, cadmium bromide, cadmium chloride, cadmium iodide, cadmium nitrate, chromium bromide, chromium chloride, chromium iodide, cesium bromide, cesium chloride, sodium bromide, sodium iodide, sodium nitrate, sodium nitrite, potassium iodide, potassium nitrate, manganese bromide, manganese chloride, zinc bromide, zinc chloride, zinc iodide, sodium monofluoroacetate, sodium trifluoroacetate, sodium 3-fluoropropionate, potassium monofluoroacetate, potassium trifluoroacetate, potassium 3-fluoropropionate); U.S. Pat. No. 7,921,910 (e.g., the lanthanide series of rare earth metals, strontium, barium, gallium, germanium, and combinations thereof, particularly, lanthanum, cerium, strontium, barium, gallium, germanium, tantalum, zirconium, vanadium, chromium, manganese, and combinations thereof, especially lanthanum, cerium, and combinations thereof, ZrSiO₄, ZnO, SrO(CO₂), Nd₂O₅, Pr₆O₁₁, MnO, CuO, Cr₂O₃, NiO, V₂O₅, Co₃O₄, Sb₂O₃, La₂O₃, CeO₂); and U.S. Pat. No. 6,991,780 (e.g., aluminum-zirconium antiperspirant salt compositions such as Zr(OH)_{4-b}X_{b} wherein X is Cl, Br, I, or NO₃; and b is about 0.7 to about 4.0), all of which are incorporated herein). Methods and apparatuses for detecting the tracer elements in accordance with the present invention include the systems, devices, methods, and apparatuses such as inductively-coupled plasma (ICP), X-ray fluorescence, or proton-induced X-ray emission (PIXE), chemical analysis, etc.

In another and/or alternative aspect of the present invention, the tracer element can optionally be in the form of one or more nanomaterials and/or types of nanomaterials such as, but not limited to, nanotubes, nanocomposites, nanohorns, functionalized nanotubes, metalized nanotubes, combinations of different nanomaterials, and combinations of different functionalized nanotubes and/or metalized nanotubes, e.g., functionalized nanotubes as disclosed in U.S. Pat. No. 7,858,691 (e.g., carbon nanotubes surface functionalized with oxygen-bearing molecules); U.S. Pat. No. 7,854,945 (e.g., functionalized carbon nanotubes); U.S. Pat. No. 8,062,702 (e.g., coated fullerene comprising a layer of at least one inorganic material covering at least a portion of at least one surface of a fullerene; and at least one composite matrix selected from the group consisting of polymers, ceramics and inorganic oxides); U.S. Pat. No. 7,968,489 (Carbon nanotubes, also known as fibrils, are vermicular carbon deposits having diameters less than 1.0μ, generally less than 0.5μ, and typically less than 0.2μ. Carbon nanotubes can be either multi walled [i.e., have more than one graphene layer more or less parallel the nanotube axis] or single walled [i.e., have only a single graphene layer parallel to the nanotube axis]. Other types of carbon nanotubes are also known, such as fishbone fibrils [e.g., wherein the graphene layers exhibit a herringbone pattern with respect to the tube axis], etc. Carbon nanotubes may be in the form of discrete nanotubes, aggregates of nanotubes [i.e., dense, microscopic particulate structure comprising entangled carbon nanotubes] or a mixture of both. Carbon nanotubes are distinguishable from commercially available continuous carbon fibers. Carbon fibers have aspect ratios (L/D) of at least 10⁴ and often 10⁶ or more, while carbon nanotubes have desirably large, but unavoidably finite, aspect ratios [e.g., less than or greater than 100]. The diameter of continuous carbon fibers, which is always greater than 1.0μ and typically 5 to 7μ, is also far larger than that of carbon nanotubes, which is usually less than 1.0μ. Carbon nanotubes also have vastly superior strength and conductivity than carbon fibers.); U.S. Pat. No. 6,905,667 (carbon nanotube surfaces are functionalized in a non-wrapping fashion by functional conjugated polymers that include functional groups. The polymers can be noncovalently bonded with carbon nanotubes in a non-wrapping fashion. The polymers can be provided having a relatively rigid backbone that is suitable for noncovalently bonding with a carbon nanotube substantially along the nanotube's length, as opposed to about its diameter. Examples of rigid functional conjugated polymers that may be utilized in embodiments of the present invention include, without limitation, poly(aryleneethynylene)s and poly(3-decylthiophene). The polymers can comprise at least one functional extension from the backbone for functionalizing the nanotube.); U.S. Pat. No. 7,771,696 (A composition is provided in which carbon nanofibers are functionalized with at least one moiety where the moiety or moieties comprise at least one bivalent radical. The composition can include a nanocomposite, such as polyimide films. Carbon nanofiber (CNF) includes all varieties of carbon nanofibers, including all types of internal and external structures. Examples of internal structures include, but are not limited to, arrangement of the graphene layers as concentric cylinders, stacked coins, segmented structures, and nested truncated cones. Examples of external structure include, but are not limited to, kinked and branched structures, amount and extent of surface rugosity, diameter variation, nanohorns, and nanocones. CNFs also include structures that have a hollow interior and those that do not. The hollow core, if it exists, can have a diameter of 20 and above, or 20-490 nm, or 30-190 nm, or 50-190 nm, or 50-90 nm. CNFs can have an outer diameter dimension of 30 nm and above, or 30-500 nm, or 40-200 nm, or 60-200 nm, or 60-100 nm. Aspect ratios for CNFs can be 500 and above, or 800 and above, or 1000 and above.); U.S. Pat. No. 7,459,137 (Functionalizing carbon nanotubes by reacting them with organic functionalizing agents in the absence of solvent [“solvent-free” conditions]. Carbon nanotubes can comprise both multi- and single-wall varieties. They can be produced by any known technique and can be of any length, diameter, or chirality which suitably provides for carbon nanotubes functionalized under solvent-free conditions. Samples of carbon nanotubes can comprise a range of lengths, diameters, and chiralities, or the nanotubes within the sample may be largely uniform. The samples may also be in the form of “ropes” or macroscopic mats called “bucky paper”. Functionalization comprises attaching organic and/or organometallic moieties to the carbon nanotubes at their ends, their sidewalls, or both. Generally, this functionalization involves a covalent bond between the functional moiety and the carbon nanotube and it is accomplished by reacting the carbon nanotubes with an organic functionalizing agent. An organic functionalizing agent may be any species that suitably functionalizes carbon nanotubes under solvent-free conditions. Organic functionalizing agents include, but are not limited to, diazonium species; aryl radicals; alkyl radicals; aryl carbocations; aryl carbanions; alkyl carbanions; alkyl carbocations; 1,3-dipoles; carbenes; heteroatom-containing radicals, cations, and anions; ylides; benzyne; dienes; dienophiles, and combinations thereof. Organic functionalizing agents may further include organometallic species such as organozincates, carbenes, Grignard reagents, Gillman reagents, organolithium reagents, and combinations thereof.); U.S. Pat. No. 7,241,496 (Carbon nanotube surfaces are functionalized in a non-wrapping fashion by functional conjugated polymers that include functional groups. Polymers that are non-covalently bonded with carbon nanotubes in a non-wrapping fashion can be used. Polymers can be provided that comprise a relatively rigid backbone that is suitable for non-covalently bonding with a carbon nanotube substantially along the nanotube's length, as opposed to about its diameter. The major interaction between the polymer backbone and the nanotube surface can be parallel π-stacking. The polymers can comprise at least one functional extension from the backbone that are any of various desired functional groups for functionalizing a carbon nanotube. Carbon nanotubes are elongated tubular bodies which are typically only a few atoms in circumference. The carbon nanotubes are hollow and have a linear fullerene structure. The length of the carbon nanotubes potentially may be millions of times greater than their molecular-sized diameter. Both single-walled carbon nanotubes (SWNTs), as well as multi-walled carbon nanotubes (MWNTs) can be used); U.S. Pat. No. 6,203,814 (Graphitic nanotubes, which includes tubular fullerenes (commonly called “buckytubes”) and fibrils, are functionalized by chemical substitution or by adsorption of functional moieties. The graphitic nanotubes which are uniformly or non-uniformly substituted with chemical moieties or upon which certain cyclic compounds are adsorbed and to complex structures comprised of such functionalized fibrils linked to one another.); U.S. Pat. No. 8,058,364 (Free-radical addition reactions which graft [i.e., chemically bond] molecules onto the nanoscale fibers' surfaces with minimal effect on the mechanical properties of the nanoscale fibers themselves. “Chemically bonded” or “chemical bond” refers to covalent bonds or ionic bonds between molecules and the atoms on the nanoscale fibers' surfaces resulting from a chemical reaction of the molecules and the atoms on the nanoscale fibers' surfaces. Examples of chemical bonds include covalent bonds and ionic bonds such as negatively charged SWNT/Li⁺ bonding.); and U.S. Pat. No. 7,976,816 (Functionalizing the wall of single-wall or multi-wall carbon nanotubes by use of acyl peroxides to generate carbon-centered free radicals to allow for the chemical attachment of a variety of functional groups to the wall or end cap of carbon nanotubes through covalent carbon bonds without destroying the wall or endcap structure of the nanotube. Carbon-centered radicals generated from acyl peroxides can have terminal functional groups that provide sites for further reaction with other compounds. Organic groups with terminal carboxylic acid functionality can be converted to an acyl chloride and further reacted with an amine to form an amide or with a diamine to form an amide with terminal amine. The reactive functional groups attached to the nanotubes provide improved solvent dispersibility and provide reaction sites for monomers for incorporation in polymer structures. The nanotubes can also be functionalized by generating free radicals from organic sulfoxides. Sidewall functionalizing of single-wall carbon nanotube comprises decomposing a diacyl peroxide in the presence of carbon nanotubes wherein the decomposition generates carbon-centered free radicals that react and form covalent bonds with carbon in the single-wall carbon nanotube wall to form a single-wall carbon nanotube sidewall functionalized with at least one organic group through a carbon bond to the nanotube. An acyl peroxide, also known as a diacyl peroxide, is a compound with a structure of the type RC(O)OOC(O)R′, where R and R′ groups can be either alkyl or aryl. The acyl peroxide can be an aroyl peroxide wherein the R or R′ group comprises an aromatic component. The acyl peroxide can be an aroyl peroxide and comprises benzoyl peroxide, which, upon decomposition, liberates carbon dioxide and generates phenyl radicals that attach to the sidewalls of the nanotubes to form sidewall phenylated single-wall carbon nanotubes.), all of which are incorporated herein.

In another and/or alternative aspect of the present invention, the tracer element can be an identifier tag that includes one or more RFID, MRD, and/or other tag-device. A variety of RFID devices are disclosed in US Publication No. 2010/0007469. (The nano RFID device or tag may be less than about 150 nanometers in size. The nano RFID device may be a passive, active or semi-passive nano RFID device. The nano RFID device may include a nano antenna that may comprise one or more carbon tubes. The nano RFID device may include a nano battery. The nano RFID device may include an environmentally-reactive layer that reacts to its immediate environment to affix or adhere to a target. Most common RFID tags typically contain at least two parts. One is an integrated circuit for storing and processing information, modulating and demodulating a radio frequency (RF) signal, and other specialized functions. The second part is an antenna for receiving and transmitting a signal. A technology called “chipless RFID” allows for discrete identification of tags without an integrated circuit, thereby allowing tags to be printed directly onto assets at a lower cost than traditional tags. Passive RFID tags typically have no internal power supply. The electrical current induced in the antenna by the incoming radio frequency signal provides just enough power for the CMOS integrated circuit in the tag to power up and transmit a response. Most passive tags signal by backscattering a carrier wave from a reader. This may mean that the antenna has to be designed both to collect power from the incoming signal and also to transmit the outbound backscatter signal. The response of a passive RFID tag is not necessarily just an ID number; the tag chip can contain non-volatile, perhaps writable, EEPROM for storing data. Semi-passive tags are similar to active tags in that they have a power source, but it may only power the micro-circuitry and may not power the broadcasting of the signal. The response may be powered by the backscattering of the RF energy from the reader.); US Publication No. 2010/0001841 (An RFID device (RFID tag) of about 150 nanometers or smaller in dimension. The RFID device may include semiconductors as small as is 90-nm, perhaps with some chips configured and provided at the 65-nm, 45-nm and/or 30-nm size level. The technology for the included electrical circuitry may include CMOS or related technology for low power consumption. A nano RFID device constructed by nanotechnology techniques provides advantages over the currently available RFID devices such as permitting the RFID device to be distributed by airborne, ingestion, or contact distribution (perhaps by aerosol or a mist, for example), or constructed to react to an specific environmental factor for embedded/affixing to a surface or specific type of material (e.g., an organic material). This provides for dynamic distribution of the RFID device to track targeted subjects or objects.); and US Publication No. 2010/0001846, all of which are incorporated fully herein, can be used as the tracer element. A variety of micro-resonant devices are disclosed in US Publication No. 2009/0027280 (A micro-resonant device (MRD) that generates resonance at radio frequencies. These individual, often monolithic, devices can be located in three-dimensional space and tracked anywhere in a target area using a conventional MRI scanner or other transducers, e.g., radiofrequency transducers. The MRDs generate high-sensitivity contrast in conventional MRI scanners, have a diameter of anywhere from a few nanometers to 1000 microns, and can be manufactured using micro-electro-mechanical systems (MEMS) technology. The devices are optionally coated to isolate them from the environment. The monolithic MRDs can include an antenna component that receives an excitation signal and transmits an emission signal; and a resonator component that receives an excitation signal and generates a corresponding emission signal; and, optionally an outer coating that envelopes the device and isolates the device from its environment. These devices have an overall diameter of less than about 1000 microns, e.g., 100 or 10 microns, and a Q value of greater than about 5, e.g., greater than 10, 50, 100, or much higher, and the emission signal is (i) a resonant frequency of the device emitted at a delayed time compared to the excitation signal (or at a time after the excitation signal has stopped), (ii) a frequency different than the excitation signal; (iii) a signal at a different polarization than the excitation signal, or (iv) a resonant frequency of the device (when the device is tuned to the same frequency as the nuclei being imaged) which upon excitation by an excitation field (e.g., a magnetic field), distorts the applied excitation field. The antenna component and the resonator component can be the same component, i.e., one component that functions as both an antenna and as a resonator. When the coating is present, the coating can be cross-linked, and the carbon can be or include amorphous carbon, diamond, or nano-crystalline diamond. The MRDs can be designed such that the resonant frequency is proportional to an applied magnetic field, e.g., by fabricating the resonator of a magnetic metal or alloy to induce magnetic field dependence to the resonant frequency. The MRD can be in the form of cylindrical or prismatic length extender bars that include a transducer material, e.g., a piezoelectric or magnetostrictive transducer material, and that have a length of less than about 100 microns and a diameter of less than about 100 microns; and optionally an outer coating that envelopes the device and isolates the device from its environment. The MRD can resonate at a resonant frequency of greater than about 50 MHz after receiving an excitation signal at the resonant frequency. The resonant frequency can be greater than about 400 MHz, greater than about 2 GHz, or even greater than 1 THz. The MRD can be in the form of devices that include a hermetically-sealed housing having walls forming an internal chamber, a cantilever arranged within the internal chamber and having a free end and a fixed end connected to a wall of the housing, and an electrode arranged within the internal chamber in parallel and spaced from the cantilever; wherein the overall size of the device is no larger than about 1000 microns, e.g., no larger than 100 or 10 microns. The cantilever and the electrode can each be made of silicon (e.g., polysilicon) and the housing can include silicon nitride. The cantilever and electrode can be made of the same material, or different materials, e.g., with different electron work functions. For example, one material of the cantilever or electrode can be silicon doped N and a second material of the electrode or cantilever can be silicon doped P. The cantilever can be made of a magnetic metal or alloy to induce magnetic field dependence to the resonant frequency. The MRD can be in the form of a sandwich of at least two layers rolled into a cylinder, wherein a first layer includes a conductor and a second layer comprises an insulator; wherein the device has an overall diameter of less than 5 mm and a Q value of greater than 5 and wherein, when exposed to an excitation signal at a resonant frequency of the device, the device generates an emission signal comprising the resonant frequency for a time after the excitation signal has ended. The MRD can include a third magnetic layer made of, e.g., iron, nickel, cobalt, or alloys thereof, or other magnetic materials described herein. The MRD can include an outer coating that envelopes the device and isolates the device from its environment. The MRD can be in the form of planar L-C resonator devices that include a spiral inductor and a thin-film capacitor. The new MRD can be manufactured in the form of piezoelectric cantilever resonator devices having a loop antenna. The MRD can be tracked by generating an excitation signal in a target area in which the device might be located; receiving an emission signal from the one or more MRDs, if any, in the target area; and processing the emission signal to determine the location of the device. The MRD can be imaged by processing the emission signal and generating an image from the processed emission signal. The MRDs can have an overall diameter of about 10 microns or less. The emission signal can be a resonant frequency of the MRD, and the device can further include a magnetic material to induce magnetic field dependence to the resonant frequency. The emission signal can be a frequency of at least 100 MHz, e.g., 400 MHz, 2 GHz, or 1 or more THz. The MRD can be attached to an object and be used to track the object within a target area. The MRD can include one or more ligands that specifically bind to a target moiety and induce a change in the frequency of the emission signal of the MRD to sense a change in the environment of the target area. The MRD can have an overall outer diameter or dimension of less than about 1000 microns, and can be much smaller, e.g., less than 500, 250, 100, 50, 20, 10, 5, or 1 micron, or even on the nanometer scale, e.g., 500, 250, 200, 100, 50, 25, 10, or 5 nanometers. The MRD can be individual, standalone, monolithic devices, or can be made of a set of nano-resonant devices that are each on the nanoscale, i.e., about 500 nanometers or less, e.g., less than 250, 100, 50, 25, 10, or 5 nanometers in size. The nano-resonant device can either (i) individually produce a resonant signal, and when acting in concert in a particular target location, the set of nano-resonant devices produces a collective signal of sufficient power to be detected in the same way that a signal from a micro-resonant device is detected, or (ii) individually do not produce a signal, but assemble, e.g., self-assemble, at a target location to form a MRD to produce a detectable signal or collectively act like a micro-resonant device to produce a detectable signal. The nano-resonant device can produce a detectable signal and serve as a micro-resonant device, depending on its size and resonant frequency. The MRD can be a passive, robust, solid-state device. The MRD can be designed and fabricated so that its resonant frequency is sensitive to its surrounding temperature, chemistry, pH, or specific target moieties, such as specific ions or chemicals, thus making it useful as local sensors with an RF readout. The MRD can be composed of metallic layers can be detected by conventional computed tomography (CT). The MRDs can act as RF tags to track the MRD.); and US Publication No. 2009/0027280, all of which are incorporated fully herein) can be used as the tracer element. A variety of nano-devices including nano-robots are disclosed in U.S. Pat. No. 8,269,648 (A system for communicating information to nano sensors located within a select subsurface region can be provided wherein a plurality of transmit antennae located at multiple positions on or below the terrain surface, the antennae adapted to transmit immediately in the far field electromagnetic energy beam signals from multiple positions on or below the terrain surface and separated from the select subsurface region via geological strata, the electromagnetic energy beam signals of a predetermined frequency, duration, and power that combine to cover a target area of the select sub surface region; and a plurality of nano sensors located in an oil reservoir at the select subsurface region and responsive to said electromagnetic beam signals to activate a function of the nano sensors. The system can comprise a plurality of receive antennae adapted to receive reflections from the target area in response to the transmitted energy beam signals impinging thereon, wherein the nano sensors are adapted to reflect or absorb the particular frequencies transmitted by the antennae such that the reflections are characteristic of the nano sensors located within the target area being impinged upon by the transmitted far field electromagnetic energy beam signals. Each of the transmit antennae can comprise a compact parametric antenna having a dielectric, magnetically-active, open circuit mass core, ampere windings around said mass core, said mass core being made of magnetically chemical additive having a capacitive electric permittivity from about 2 to about 80, an initial permeability from about 5 to about 10,000 and a particle size from about 2 to about 100 micrometers; and an electromagnetic source for driving said windings to produce an electromagnetic wavefront. A communications method can be provided for communicating information to nano sensors located within a select subsurface region: from multiple positions on or below the terrain surface and separated from the select subsurface region via geological strata, transmitting immediately in the far field electromagnetic energy beam signals of a predetermined frequency, duration, and power that combine to cover a target area of the select sub surface region; and receiving via one or more nano sensors located in an oil reservoir at the select subsurface region said electromagnetic beam signals, wherein the one or more nano sensors are responsive to the received electromagnetic beam signals to activate a function of the nano sensors. The nano sensors can be responsive to the received electromagnetic beam signals to recharge a battery of the nano sensors using the received electromagnetic energy signals. The nano sensors can be responsive to the received electromagnetic beam signals to realign themselves according to the magnetic field impinging thereon. The nano sensors can be responsive to the received electromagnetic beam signals to effect a chemical reaction within the oil reservoir. In another embodiment, the nano sensors are responsive to the received electromagnetic beam signals for initiating communications with other said nano sensors. The nano sensors can be responsive to the received electromagnetic beam signals for retrieving information from memory contained within the nano sensors and transmitting the information. The nano devices can receive the transmitted electromagnetic energy to recharge a power system within the nano devices. The nano devices can be designed to reflect a portion of the energy from the transmissions, wherein the reflected energy related to relative changes in the position of an ensemble of nano devices existing in a given location. A source of electromagnetic energy from an array of antennae transmitting immediately in the far field is provided for imparting pulses, wherein the pulses will be reflected by the nano devices according to the reflectivity to the nano devices material and its location as it may exist. An array of receiver antennae may be used to initially establish a reference of the reflected pattern, and then operated in conjunction with the transmit array to monitor the movement of the nano devices. A source of the electromagnetic energy from an array of antennae transmitting in the far field can be provided for triggering or activating nano devices. A source of electromagnetic energy from an array of antennae transmitting immediately in the far field can be provided for imparting pulses at the depth of the fluid reservoir whereby the returns reflected by nano devices according to the reflectivity to the nano particle or nano sensor material and its location for mapping a 3-dimensional map and over time a 4-dimensional map. A source of electromagnetic energy from an array of antennae transmitting in the far field can be provided for imparting pulses to communicate with nano devices to effect motion of the nano devices.), which is incorporated fully herein, can be used as the tracer element.

In another and/or alternative aspect of the present invention, the tracer element includes one or tracer molecules, and/or one or more different tracer molecules. The one or more tracer molecules can include at least one of fluorescent molecules, UV-active molecules, isotopically-enriched molecules (e.g., molecules having mass spectra distinct from non-isotopically enriched molecules, etc.), radiolabeled molecules, radioactive molecules, metal nanoparticles, hydrophobic molecules, hydrophilic molecules, rare earth nanoparticles, phosphorescent or fluorescent nanoparticles, stable isotopes, and combinations thereof. Easily ionizable molecules such as, but not limited to, halogen-containing molecules can also be used as tracer molecules due to their low detection threshold. Fluorinated compounds can be used as tracer materials. Sulfonated compounds can be used as tracer materials. Triheptylamine (THA) can be used as a tracer material. THA is a highly hydrophobic molecule due to its long alkyl chains. Furthermore, THA's nitrogen atoms can be easily distinguished by mass spectrometry according to the nitrogen rule, where an odd number of nitrogen atoms will afford an odd mass. The one or more tracer molecules can also include fluorescent dyes, such as 1,5-diphenyloxazole or fluorescein. In one non-limiting embodiment, the one or more releasable tracer molecules can be non-isotopically enriched molecules that are easily detectable by their mass spectra or other unique spectroscopic signature; however, this is not required. In another non-limiting embodiment, the releasable tracer molecules can include metal nanoparticles and molecules that are sensitive to the presence of heavy metals (e.g., chelating ligands, etc.); however, this is not required. In another non-limiting embodiment, the releasable tracer molecules can include molecules that are non-radioactive; however, this is not required. In another non-limiting embodiment, the releasable tracer molecules can include molecules that are radioactive and thus detectable by a scintillation counter; however, this is not required.

In another and/or alternative aspect of the present invention, the tracer element can be incorporated into degradable materials by encapsulation (e.g., polymer, etc.); however, this is not required. The encapsulation of the tracer element (when used) can be used to create controlled release detection and/or allow placement and then release at different depths and within a well formation. For example, one or more tracer elements can be coated with a dissolvable material (e.g., polymer, metal, carbohydrate, sugar, etc.) prior to or after being applied onto or incorporated into a degradable component. The coating is generally formulated to dissolve when exposed to certain environmental conditions (e.g., fluid temperature, fluid composition, etc.).

In another and/or alternative aspect of the present invention, there is provided a system for assuring performance of a degradable component that includes a) a degradable material partially or fully forming the degradable component; and b) one or more tracer elements and/or types of tracer elements incorporated on and/or in the degradable component, wherein the one or more tracer elements are configured to be released from the degradable component when the degradable component partially or fully degrades.

In another and/or alternative aspect of the present invention, there is provided a method of detecting one or more tracer elements and/or types of tracer elements at some distance from a location of a degradable component (e.g., a tool, a component of a tool, valve, plug, ball, frac ball, pipe, sleeve, casting, etc.).

In another and/or alternative aspect of the present invention, there is provided a tracer element that is a stable isotope/element that is incorporated on and/or in a degradable component which is detectable using analytical techniques prior to, during, and/or after the degradable component partially or fully degrades.

In another and/or alternative aspect of the present invention, there is provided a tracer element that is an oxide or other type of compound (such as a rare earth oxide) that is incorporated on and/or in a degradable component which is detectable using analytical techniques prior to, during, and/or after the degradable component partially or fully degrades. The average particle size of the compound can be no more than about 10 microns, and typically no more than about 1 micron, and typically no more than about 0.5 micron. The compound can be formulated or be designed to be detectable by various techniques (e.g., detection of highly polar molecules or a radioisotope, an isotope that can be activated, UV-chemical additive, a fluorescent material, die or phosphorescent particles, etc.).

In another and/or alternative aspect of the present invention, there is provided a tracer element that is a rare earth material not normally found in the formation fluids that is incorporated on and/or in a degradable component which is detectable using analytical techniques prior to, during and/or after the degradable component partially or fully degrades.

In another and/or alternative aspect of the present invention, there is provided a tracer element that is incorporated uniformly throughout the degradable component.

In another and/or alternative aspect of the present invention, there is provided a tracer element that is located on and/or within one or more specific areas or regions of the degradable component.

In another and/or alternative aspect of the present invention, there is provided a degradable component having one or more cavities that are formed by machining and then plugged, and wherein the tracer element is positioned in the plugged cavity. The cavity and optional sealing structure for the cavity can be configured to release a portion or all of the tracer element from the cavity after the degradable component has partially or fully degraded (e.g., 5%-100% degradation and all values and ranges therebewteen). Generally, the cavity of the degradable component is not designed to allow release of said tracer elements from the cavity until the degradable component has degraded at least about 10%, and typically at least about 20%, and more typically at least about 20-60%. Generally, the size of the cavity is no more than 80 vol. % of the degradable component, and typically about 0.1-80 vol. % (and all values and ranges therebetween) of the degradable component, and more typically about 0.5-60 vol. % of the degradable component, and more typically about 0.75-45 vol. % of the degradable component.

In another and/or alternative aspect of the present invention, there is provided a degradable component having one or more cavities that can be closed by use of a plug, wherein said plug is connected to the cavity by a threaded connection, interference fit, swaged connection, etc. The plug may or may not be formed of a degradable material.

In another and/or alternative aspect of the present invention, there is provided a degradable component having one or more cavities wherein at least one of the cavities includes one or more tracer elements in an amount of at least about 0.01 grams. In one non-limiting embodiment, at least one of the cavities includes one or more tracer elements in an amount of 0.01-10 grams (and all values and ranges therebetween).

In another and/or alternative aspect of the present invention, there is provided a tracer element that is in the form of an RFID tag, magnetic wire, or other information carrying device that is incorporated on and/or in a degradable component which is detectable using analytical techniques prior to, during, and/or after the degradable component partially or fully degrades.

In another and/or alternative aspect of the present invention, there is provided a tracer element that is in the form of a tracer molecule or element that is incorporated on and/or in a degradable component which is detectable using analytical techniques prior to, during, and/or after the degradable component partially or fully degrades.

In another and/or alternative aspect of the present invention, there is provided a degradable component that includes a plurality of tracer elements, and wherein the plurality of tracer elements is the same.

In another and/or alternative aspect of the present invention, there is provided a degradable component that includes a plurality of tracer elements, and wherein some of the tracer elements are different from some of the other tracer elements.

In another and/or alternative aspect of the present invention, there is provided a method for determining 1) whether a degradable component has begun to degrade, 2) the degree to which the degradable component has degraded, 3) whether the one or more particular regions of the degradable component has begun to degrade and/or the degree to which such one or more particular regions have degraded, and/or 4) whether the degradable component has been sufficiently removed from a location (e.g., a location in a well, etc.), wherein such method comprises the steps of a) providing a degradable component (e.g., a tool, component of a tool, valve, plug, ball, frac ball, etc.); b) providing one or more tracer elements (and if a plurality of tracer elements, the tracer elements can be the same or different) that are incorporated on and/or in the degradable component, wherein the one or more tracer elements are configured to be released from the degradable component when the degradable component partially or fully degrades; c) exposing the degradable component to fluid (e.g., a flowing stream of fluid, etc.) which causes the degradable component to partially or fully degrade and thereby partially or fully releasing one or more tracers element from the degradable component that previously located on and/or in the degradable component; and d) providing a detection arrangement (e.g., sensor, testing lab, visual inspection, etc.) to detect the presence and/or concentration of one or more tracer elements prior to release from the degradable component and/or after released from the degradable component due to the partial or full degradation of the degradable component. The sensor can be located at a location away from the degradable component (e.g., one or more feet to one or more miles from the location of the degradable component [and all values and ranges of such distances therebetween]); however, this is not required. For example, if fluid (e.g., water, salt solution, brine, polymer solution, etc.) is flowed into a location of the degradable component (e.g., well, etc.), the flowing fluid can be used to cause the degradable component to partially or fully degrade, thereby causing one or more tracer elements to be released from the degradable component and to flow with the fluid downstream from the degradable component. At some location downstream of the degradable component, the fluid can be 1) analyzed by a sensor to detect the presence of one or more tracer elements; 2) samples of the fluid can be taken and tested by a sensor, some other testing device, or in a lab to test and/or detect the presence of one or more tracer elements; and/or 3) visually detected (e.g., a person seeing a color change in the fluid, etc.) to thereby detect the presence of one or more tracer elements. The detection of the presence of a tracer element and/or the amount of detected tracer element can be used to determine that 1) the degradable component has not degraded, 2) the degradable component has not sufficiently degraded, 3) a certain portion of the degradable component has not degraded, 4) a certain portion of the degradable component has begun to degrade, 5) the degree of degradation of one or more portions of the degradable component, and/or 6) the degradable component has sufficiently degraded. As can be appreciated, the method can be used to determine the degradation status of a plurality of degradable components that are the same or different. Different tracer elements can be used to differentiate the degree of degradation of different components and/or different regions of components.

In another and/or alternative aspect of the present invention, the detection arrangement can be located at the surface of a well site and/or on the surface above the location of the degradable component that includes the one or more tracer elements.

In another and/or alternative aspect of the present invention, the detection arrangement can be located remotely from the degradable component (e.g., one or more feet to one or more miles from the location of the degradable component [and all values and ranges of such distances therebetween]).

In another and/or alternative aspect of the present invention, the composition of the fluid and/or the flowrate of the fluid to which the degradable component is exposed can be used to 1) control a rate of degradation of the degradable component, 2) measure or estimate dissolution rates of the degradable component, 3) measure or estimate the degree of degradation of the degradable component, and/or 4) measure or estimate flow rates of fluids through specific fluid or zones in a well due to the progression of dissolution or degradation of the degradable component. As such, tracer elements that are the same or different from the one or more tracer elements in the degradable component can be inserted into the fluid so as to determine 1) if the fluid has encountered one or more of the degradable components, 2) the flow rate of the fluid about the one or more of the degradable components, and/or the flow of fluid through one or more regions of a well.

In another and/or alternative aspect of the present invention, a detection arrangement is used to assure that a degradable component has properly degraded by discretely locating the tracer element on and/or in one or more locations of the degradable component, detecting the presence of the tracer element, and/or by estimating the total amount of the tracer element released from the degradable component.

In another and/or alternative aspect of the present invention, the degradable component can be formed of a plastic material or metal material (magnesium, magnesium alloy, aluminum alloy, etc.) which may or may not include a coating material and/or one or more additives. Non-limiting examples of degradable metal materials are disclosed in US Publication No. 2015/0239795 (Magnesium alloy that contains at least at least 30 wt. % magnesium, typically greater than 50 wt. %, and more typically at least about 70 wt. %. The metals that can be included in the magnesium alloy can include, but are not limited to, aluminum, calcium, lithium, manganese, rare earth metal, silicon, SiC, yttrium, zirconium and/or zinc. Non-limiting examples of metals or metal alloys other than magnesium that are degradable metal alloys include aluminum alloys (e.g., aluminum alloys including 75+wt. % aluminum and one or more of bismuth, copper, gallium, magnesium, indium, silicon, tin, and/or zinc); calcium; Ca—Mg, Ca—Al; and Ca—Zn.); US Publication No. 2015/0299838 (magnesium or magnesium alloy constitutes about 50.1 wt. % to 99.9 wt. % of the magnesium composite and one or more additives such as copper, nickel, cobalt, titanium, iron, wherein the one or more additives generally have an average particle diameter size of at least about 0.1 microns, typically no more than about 500 microns and a higher melting point that magnesium.); and US Publication No. 2015/0240337 (A metal cast structure wherein the grain boundary composition and the size and/or shape of the insoluble phase additions can be used to control the dissolution rate of such composite. The composition of the grain boundary layer can optionally include two added insoluble particles having a different composition with different galvanic potentials, either more anodic or more cathodic as compared to the base metal or base metal alloy. The base metal or base metal alloy can include magnesium, zinc, titanium, aluminum, iron, or any combination or alloys thereof. The added insoluble particles that have a more anodic potential than the base metal or base metal alloy can optionally include beryllium, magnesium, aluminum, zinc, cadmium, iron, tin, copper, and any combinations and/or alloys thereof. The insoluble particles that have a more cathodic potential than the base metal or base metal alloy can optionally include iron, copper, titanium, zinc, tin, cadmium lead, nickel, carbon, boron carbide, and any combinations and/or alloys thereof. The grain boundary layer can optionally include an added component that is more cathodic as compared to the base metal or base metal alloy. The composition of the grain boundary layer can optionally include an added component that is more cathodic as compared to the major component of the grain boundary composition. The grain boundary composition can be magnesium, zinc, titanium, aluminum, iron, or any combination of any alloys thereof. The composition of the grain boundary layer can optionally include an added component that is more cathodic as compared to the major component of the grain boundary composition and the major component of the grain boundary composition can be more anodic than the grain composition. The cathodic components or anodic components can be compatible with the base metal or base metal alloy in that the cathodic components or anodic components can have solubility limits and/or do not form compounds. The component (anodic component or cathodic component) can optionally have a solubility in the base metal or base metal alloy of less than about 5% (e.g., 0.01-4.99% and all values and ranges therebetween), typically less than about 1%, and more typically less than about 0.5%. The composition of the cathodic components or anodic components in the grain boundary can be compatible with the major grain boundary material in that the cathodic components or anodic components have solubility limits and/or do not form compounds. The strength of metal cast structure can optionally be increased using deformation processing and a change dissolution rate of less than about 20% (e.g., 0.01-19.99% and all values and ranges therebetween), typically less than about 10%, and more typically less than about 5%. The ductility of the metal cast structure can optionally be increased using nanoparticle cathode additions. The metal cast structure can optionally include chopped fibers.); all of which are incorporated herein by reference. A non-limiting example of degradable plastic or polymer materials is disclosed in US 2016/0137912 (The expandable composite material can include one or more polymer materials selected from the group consisting of polyacetals, polysulfones, polyurea, epoxys, silanes, carbosilanes, silicone, polyarylate, and polyimide. The expandable material can include one or more materials selected from the group consisting of Ca, Li, CaO, Li₂O, Na₂O, Fe, Al, Si, Mg, K₂O and Zn. The expandable material generally ranges in size from about 106 μm to 10 mm. The expandable composite material can include one or more catalysts for accelerating the reaction of the expandable material; however, this is not required. The catalyst can include one or more materials selected from the group consisting of AlCl₃ and a galvanically chemical additive. The expandable material can include strengthening and/or diluting fillers; however, this is not required. The strengthening and/or diluting fillers can include one or more materials selected from the group consisting of fumed silica, silica, glass fibers, carbon fibers, carbon nanotubes and other finely divided inorganic material. The expandable material can include a surface coating or protective layer that is formulated to control the timing and/or conditions under which the reaction or expanding occurs; however, this is not required. The surface coating can be formulated to dissolve when exposed to a controlled external stimulus (e.g., temperature and/or pH, chemicals, etc.). The surface coating can be used to control activation of the expanding of the core or core composite. The surface coating can include one or more materials such as, but not limited to, polyester, polyether, polyamine, polyamide, polyacetal, polyvinyl, polyureathane, epoxy, polysiloxane, polycarbosilane, polysilane, and polysulfone. The surface coating generally has a thickness of about 0.1 μm to 1 mm and any value or range therebetween.)

One non-limiting object of the present invention is the provision of a degradable component that includes one or more chemical additives that are released upon the partial or full dissolution of the degradable component, and which the one or more chemical additives are used to at least partially control the local chemical environment about the degradable component to a) maintain the rate of degradation of the degradable component, b) enhance or accelerate the degradation of the degradable component, c) delay or slow the rate of degradation of the degradable component, and/or d) offset, neutralize or remove the byproducts of the degradation of the degradable component.

In another non-limiting object of the present invention is the provision of a degradable component that includes one or more chemical additives that create a desired or proper environment for the degradable component so that the degradable component can fully or partially degrade.

In another non-limiting object of the present invention is the provision of a degradable component that includes one or more chemical additives that are 1) coated on the degradable component, 2) incorporated in the composition of the degradable component, and/or 3) contained in one or more cavities of the degradable component.

In another non-limiting object of the present invention is the provision of a degradable component that includes one or more chemical additives that are generally salts, acids, and/or buffer materials.

In another non-limiting object of the present invention is the provision of a degradable component that includes one or more chemical additives used to neutralize a high pH solution.

In another non-limiting object of the present invention is the provision of a degradable component that includes two or more different chemical additives wherein 1) the concentration of the two or more chemical additive can be the same or different, 2) the location of the two or more chemical additives on the degradable component can be the same or different, 3) the time of release of the two or more chemical additives from the degradable component can be the same or different, and/or 4) the rate of release of the two or more chemical additives from the degradable component can be the same or different.

In another non-limiting object of the present invention is the provision of a degradable component that includes one or more chemical additives that have controlled-release properties by one or more mechanisms such as a degradable or dissolvable coating about the outer surface of the chemical additive, the particle size of the chemical additive, and/or the shape of the chemical additive.

In another non-limiting object of the present invention is the provision of a degradable component that includes one or more chemical additives that can be released over a short period after exposure to the targeted depth/distance in the well and/or exposure to certain pressures, temperatures and/or chemical environment in the well.

In another non-limiting object of the present invention is the provision of a degradable component that includes one or more chemical additives that can be added in a desired amount and/or concentration into holes, or other features in the degradable component, and can be optionally covered, coated, plugged or sealed in or on the degradable component by a coating, seal, or and/or adhesive, and such optional covering, coating, plug or seal can be used to control the timing of release of the one or more chemical additives from the degradable component and/or to limit or prevent removal of the one or more chemical additives from the degradable component during handling, shipment, and placement of the degradable component in the wellbore.

In another non-limiting object of the present invention is the provision of a degradable component that includes one or more chemical additives that can be incorporated uniformly throughout the degradable component, added to specific locations on the surface of the degradable component surface, coated on the complete surface of the degradable component, placed at one or more different depths within the degradable component, and/or positioned in one or more cavities of the degradable component.

In another non-limiting object of the present invention is the provision of a degradable component that includes one or more chemical additives that are positioned in an internal cavity of the degradable component, and wherein an optional degradable plug or cap is used to control the date of release of the one or more chemical additives from the degradable component.

In another non-limiting object of the present invention is the provision of a method of influencing degradation of a degradable component comprising a) providing a degradable component (e.g., tool, device, frac ball, valve, plug, etc.) that is at least partially formed of a degradable material; b) providing one or more chemical additives that are i) coated on the degradable component, ii) incorporated in the composition of the degradable component, and/or iii) contained in one or more cavities of the degradable component, said one or more chemical additives selected to influence degradation of said degradable component while said degradable component in in a wellbore; c) placing said degradable component in the wellbore; d) providing a wellbore fluid in a region about said degradable component, said wellbore fluid contacting said degradable component while said degradable component is in the wellbore; and e) at least partially releasing the one or more chemical additives from the degradable component while the degradable component is in the wellbore to affect the salinity, pH, viscosity and/or some other fluid property of the wellbore fluid that is in contact with the degradable component to thereby influence degradation of said degradable component while said degradable component in in a wellbore.

In another non-limiting object of the present invention is the provision of a degradable component that includes one or more chemical additives that can be optionally released from the degradable component in a controlled manner into the local wellbore fluid environment.

In another non-limiting object of the present invention is the provision of a degradable component that includes one or more chemical additives that include an acid, buffer compound, salt, oxidizer, water rechemical additive, surfactant, and/or absorbent material.

In another non-limiting object of the present invention is the provision of a degradable component that includes one or more chemical additives that are formulated to partially or fully neutralize the formation of hydroxides in the wellbore fluid and/or to maintain a pH of the wellbore fluid below 10 that is located about the degradable compound.

In another non-limiting object of the present invention is the provision of a degradable component that includes one or more chemical additives that produce 1000-10000 ppm of chloride content in the wellbore fluid that is located about the degradable compound.

In another non-limiting object of the present invention is the provision of a degradable component that includes one or more chemical additives that are coated with a degradable or dissolvable material and/or be incorporated in a degradable or dissolvable matrix material to control the interaction with and/or release of the one or more chemical additives into the wellbore fluid.

In another non-limiting object of the present invention is the provision of a degradable component that includes one or more chemical additives that are in the form of granules, pellets, or powders.

In another non-limiting object of the present invention is the provision of a degradable component that includes one or more chemical additives that are incorporated in, or are in the form of, a gel, bulk scaffold, thin film, or pellet.

In another non-limiting object of the present invention is the provision of a degradable component that includes one or more chemical additives that are included or are used with an amphoteric species which is formulated to a) alter the rate of dissolution of the gel, bulk scaffold, thin film or the pellet, and/or b) alter the release of the one or more chemical additives from the gel, bulk scaffold, thin film or the pellet, thereby altering the release of the one or more chemical additives from the gel, bulk scaffold, thin film or pellet.

In another non-limiting object of the present invention is the provision of a degradable component that includes one or more chemical additives that are bound to the degradable component by a binder.

In another non-limiting object of the present invention is the provision of a degradable component that includes one or more chemical additives that are positioned on the degradable component such that the one or more chemical additives are exposed to the wellbore fluid by mechanical action such as shear, sliding, pressure pulse, etc., and/or are exposed to the wellbore fluid by dissolution of a coating or plug covering a cavity in the degradable component, and wherein such plug or coating is the same or different material as the degradable component.

In another non-limiting object of the present invention is the provision of a degradable component that includes one or more chemical additives that are included in an additional component that is connected to the degradable component such as an extension to the shoe, a mandrel extension, a lining or cylinder, etc.

In another non-limiting object of the present invention is the provision of a degradable component that includes one or more chemical additives that are added as a coating or a lining to some or all of the surface or some other region of the degradable component.

In another non-limiting object of the present invention is the provision of a degradable component that includes one or more chemical additives that are protected from damage as the degradable component is inserted into a wellbore and/or a protective coating or covering can be used to control wellbore fluid access to the one or more chemical additives on the degradable component.

In another non-limiting object of the present invention is the provision of a degradable component that includes one or more chemical additives that have been added to the degradable component while the one or more chemical additives are in a molten state.

In another non-limiting object of the present invention is the provision of a degradable component that includes one or more chemical additives wherein one or more chemical additives are or include a molten salt or acid that is added into one or more cavities in a degradable component.

In another non-limiting object of the present invention is the provision of a degradable component that includes one or more chemical additives wherein one or more chemical additives are heated to a molten state and then poured into a mold, and then the one or more chemical additives can be optionally coated with a degradable coating (e.g., PVA, PGA, PLA, PEG, cellulose, or other degradable polymer), and placed on the surface of a degradable component and/or placed on one or more cavities in a degradable component.

In another non-limiting object of the present invention is the provision of a degradable component that includes one or more chemical additives wherein the one or more chemical additives are a solid acid, such as FeCl₃, AlCl₃, or Na₂SO₄.

In another non-limiting object of the present invention is the provision of a degradable component that includes one or more chemical additives wherein the ratio of the one or more chemical additives (e.g., solid acid, etc.) to the degradable metal used in the degradable component is selected such as to shift the degradation byproducts and/or solution pH away from insoluble hydroxides to soluble sulfates or chorides or oxychlorides.

In another non-limiting object of the present invention is the provision of a degradable component that includes one or more chemical additives that are used to lower the pH of the liquid about the degradable component to less than 10, typically less than 8, more typically less than 7, and even more typically less than 6.

In another non-limiting object of the present invention is the provision of a degradable component that includes one or more chemical additives that are included on or in the degradable component are selected to ensure at least 35% solubilization (reaction) of the degradable material that partially or fully forms the degradable component, typically at least 50% solubilization (reaction) of the degradable material that partially or fully forms the degradable component, more typically at least 70% solubilization (reaction) of the degradable material that partially or fully forms the degradable component, still more typically at least 80% solubilization (reaction) of the degradable material that partially or fully forms the degradable component, yet more typically at least 90% solubilization (reaction) of the degradable material that partially or fully forms the degradable component, still yet more typically at least 95% solubilization (reaction) of the degradable material that partially or fully forms the degradable component, and even still more typically 100% solubilization (reaction) of the degradable material that partially or fully forms the degradable component.

In another non-limiting object of the present invention is the provision of a degradable component that includes one or more chemical additives that are used to inhibit or prevent the possibility of plugging and/or cementing of sand grains that would require subsequent intervention.

In another non-limiting object of the present invention is the provision of a degradable component that includes one or more chemical additives in an amount of about 35-200% (and all values and ranges therebetween) of the stoichiometric amount required to cause 30-100% of the degradable material in the degradable component to solubilize or dissolve.

In another non-limiting object of the present invention is the provision of a degradable component that includes one or more chemical additives that include two or more different salts.

In another non-limiting object of the present invention is the provision of a degradable component that includes one or more chemical additives that include two or more different salts that can be used to 1) accelerate dissolution of the degradable component, 2) reduce or eliminate the sensitivity of the degradable component to wellbore fluid salinities, and 3) enable dissolvable metals to be used in freshwater wells.

In another non-limiting object of the present invention is the provision of a degradable component that includes one or more chemical additives that include two or more different salts In one non-limiting embodiment, the chemical additive includes two or more chemical additives, such as a solid acid in the interior of the degradable component or device, and a salt closer to, or on the surface of, the degradable component or device.

In another non-limiting object of the present invention is the provision of a degradable component that includes one or more chemical additives to reduce or eliminate sensitivity of the degradable component to the chloride content present about the degradable component or device, while also creating soluble byproducts and conditions to prevent the need for subsequent intervention and wellbore cleanup.

In another non-limiting object of the present invention is the provision of a degradable component that includes one or more chemical additives to be used for the purpose of wellbore cleanup such as to remove cements, filter cakes, and deliver wellbore cleanup or gelbreaking chemistries to the wellbore.

In another non-limiting object of the present invention is the provision of a degradable component that includes one or more chemical additives that includes a solid shape of solid acid, salt or other active ingredient that is 1) coated with a degradable coating and then a) placed on and/or in a degradable component, and/or 2) mixed with the well fluid or brine in a wellbore, or 3) placed inside the degradable component (e.g., inside a degradable shell, etc.).

In another non-limiting object of the present invention is the provision of a degradable component that can be used with or formed into a degradable rubber wedge or wiper for precise control over location of release of the one or more chemical additives in the wellbore.

In another non-limiting object of the present invention is the provision of a degradable component that includes one or more chemical additives that includes a solid shape of solid acid, salt or other active ingredient that is coated with a degradable coating to provide temperature-controlled release of the one or more chemical additives in a wellbore.

In another non-limiting object of the present invention is the provision of a degradable component that can be used with or formed into a degradable rubber wedge or wiper to produce a pump-down dart, plug, or device that prevents fluid leakage around the degradable component or device until it is inserted into a desired location in the wellbore.

In another non-limiting object of the present invention is the provision of a degradable component that can be used with a slickline or wireline for precise control over location of release of the one or more chemical additives in the wellbore.

In another non-limiting object of the present invention is the provision of a degradable component that includes one or more chemical additives such as a salt, solid acid, base, active chemical, or mixture (such as a eutectic salt mixture) that can be melted and then poured into a cavity of the degradable component.

In another non-limiting object of the present invention is the provision of a degradable component that includes one or more chemical additives that are melted in its hydrated or water-containing form, and thereafter poured into a cavity of the degradable component, and then the melted one or more chemical additives are continued to be heated to remove 90-100% of the water from the one or more chemical additives so that the one or more chemical additives solidify in its anhydrous, or lower H₂O content form.

In another non-limiting object of the present invention is the provision of a degradable component that includes one or more chemical additives that are melted and poured into a cavity of the degradable component so that the degradable component does not react or dissolve while the one or more chemical additives are in a molten state, and which the one or more chemical additives do not cause significant degradation to the properties of the degradable component (less than 10% degradation of the hardness and/or strength of the degradable component) over a period of at least 1 month (e.g., 1-12 months, 1-6 months, 1-3, months) while the degradable component is stored in a non-liquid and dry conditions (less than 80 humidity) at ambient temperatures (e.g. 20-28° C.).

In another non-limiting object of the present invention is the provision of a degradable component that includes one or more chemical additives that are mixed together to form an eutectic mixture that causes a lowering of the melting point of the mixture of the two of more chemical additives such that the melted eutectic mixture can be poured into a cavity of the degradable component without causing the degradable component to melt or otherwise be damaged or deformed.

In another non-limiting object of the present invention is the provision of a degradable component that includes one or more chemical additives that cause little or no degradation to mechanical performance of the degradable component when the degradable component is placed in compression.

In another non-limiting object of the present invention is the provision of a degradable component in the form of a pill ball, plug, bridge plug, stinger, pill, frac plug in a hydraulic fracturing operation, or other dissolvable device that has a cavity that includes a solid material formed of one or more chemical additives.

In another non-limiting object of the present invention is the provision of a degradable component in the form of a pill ball, plug, bridge plug, stinger, pill, frac plug in a hydraulic fracturing operation, or other dissolvable device that contains one or more chemical additives (e.g., solid acid, salt, etc.) that enhances the rate of dissolution of the degradable component such that the degradable component is 80-100% dissolved in less than 72 hours.

In another non-limiting object of the present invention is the provision of a degradable component that include a cavity that includes one or more chemical additives that are sealed inside the degradable component by a water tight plug, interference fit, and/or polymer sealing compound.

In another non-limiting object of the present invention is the provision of a degradable component that includes a cavity that includes one or more chemical additives that are sealed inside the degradable component by a watertight plug that has the same or different degradation rate than the degradable component.

In another non-limiting object of the present invention is the provision of a degradable component that includes a cavity that includes one or more chemical additives, and which degradable component can withstand more than 5 ksi differential pressure on a seat or hydrostatic pressure of 5 ksi or more.

In another non-limiting object of the present invention is the provision of a degradable component that includes one or more tracer elements.

In another non-limiting object of the present invention is the provision of a system and method of detecting or estimating whether a degradable component has properly degraded.

In another non-limiting object of the present invention is the provision of a degradable component for use in subterranean operations wherein the degradable component includes one or more tracer elements that are released upon the partial or full dissolution of the degradable component, and which the one or more tracer elements can be detected at the surface to determine the proper removal or degradation of the degradable component.

In another non-limiting object of the present invention is the provision of a degradable component wherein the one or more tracer elements are incorporated uniformly throughout the degradable component, added to specific locations in the degradable component, or placed at different depths within the degradable component.

In another non-limiting object of the present invention is the provision of a degradable component that includes a single tracer element or different tracer elements.

In another non-limiting object of the present invention is the provision of a degradable component wherein the tracer element is uniformly dispersed in the degradable component, is located in one or more regions of the degradable component, or is concentrated in one or more regions of the degradable component.

In another non-limiting object of the present invention is the provision of a degradable component wherein the degradable component includes the addition of one or more tracer elements in an interior of the degradable component for the purpose of verifying and/or assuring that the degradable component has sufficiently degraded and/or dissolved.

In another non-limiting object of the present invention is the provision of a degradable component wherein the tracer element is less than a micron is size.

In another non-limiting object of the present invention is the provision of a degradable component wherein the type and/or amount of one or more tracer elements used in a particular degradable component is non-limiting.

In another non-limiting object of the present invention is the provision of a degradable component wherein the tracer elements can be 1) uniformly dispersed throughout a particular component, 2) concentrated in one or more regions of a particular component, and/or 3) include different types of tracer elements in different regions of a particular component.

In another non-limiting object of the present invention is the provision of a degradable component wherein the tracer element is incorporated in the degradable component and is designed to be released during or after the partial or full degradation of the degradable component.

In another non-limiting object of the present invention is the provision of a degradable component wherein one or more tracer elements are placed in an internal cavity of the degradable component and a degradable plug or cap is used to close the cavity; upon degradation of the cap or plug, the tracer elements in the cavity are partially or fully released from the cavity.

In another non-limiting object of the present invention is the provision of a degradable component wherein the tracer element is designed, after the degradable component partially or fully degrades, to release from the degradable component and be carried with fluid flow to a location at some distance from where such one or more tracer elements are released from the degradable component, and which tracer elements can be detected once such tracer elements are transported to a different location from the location of the degradable component.

In another non-limiting object of the present invention is the provision of a degradable component wherein different tracer elements are used in different regions or zones of a degradable component to provide information as to the degree to which a degradable component has degraded and/or whether a particular region of a degradable component has degraded and/or the degree to which it has been degraded.

In another non-limiting object of the present invention is the provision of a degradable component wherein different types of tracer element are incorporated and/or positioned at different regions of a degradable component.

In another non-limiting object of the present invention is the provision of a degradable component wherein different tracer elements are used in different degradable components.

In another non-limiting object of the present invention is the provision of a degradable component wherein the tracer element can be chosen from one or more microRFID, magnetic wires, nanowires, magnetic particles, fluorescing, and phosphorescent compounds and/or particles; and/or from compounds or molecules that can include stable isotopes, radioactive isotopes, rare earth or other specific elements, as well as compounds with high sensitivity in mass spectroscopy or other analytical technique that is sensitive to ppb levels.

In another non-limiting object of the present invention is the provision of a degradable component wherein the tracer element can be in the form of one or more nanomaterials and/or types of nanomaterials such as, but not limited to, nanotubes, nanocomposites, nanohorns, functionalized nanotubes, metalized nanotubes, combinations of different nanomaterials, and combinations of different functionalized nanotubes and/or metalized nanotubes.

In another non-limiting object of the present invention is the provision of a degradable component wherein the tracer element can include one or more RFID and/or other nano-device.

In another non-limiting object of the present invention is the provision of a degradable component wherein the tracer element includes one or tracer molecules and/or one or more different tracer molecules.

In another non-limiting object of the present invention is the provision of a degradable component wherein the tracer element can be incorporated into degradable materials by encapsulation to create controlled release detection and/or allow placement and then release at different depths and within a well formation.

In another non-limiting object of the present invention is the provision of a system for assuring performance of a degradable component that includes a) a degradable material partially or fully forming the degradable component, and b) one or more tracer elements and/or types of tracer elements incorporated on and/or in the degradable component, wherein the one or more tracer elements are configured to be released from the degradable component when the degradable component partially or fully degrades.

In another non-limiting object of the present invention is the provision of a method of detecting one or more tracer elements and/or types of tracer elements at some distance from a location of a degradable component.

In another non-limiting object of the present invention is the provision of a degradable component wherein the tracer element is a stable isotope/element that is incorporated on and/or in a degradable component which is detectable using analytical techniques prior to, during, and/or after the degradable component partially or fully degrades.

In another non-limiting object of the present invention is the provision of a degradable component wherein the tracer element is an oxide or other type of compound, such as a rare earth oxide, that is incorporated on and/or in a degradable component which is detectable using analytical techniques prior to, during, and/or after the degradable component partially or fully degrades.

In another non-limiting object of the present invention is the provision of a degradable component wherein the tracer element is a rare earth material not normally found in the formation fluids that is incorporated on and/or in a degradable component which is detectable using analytical techniques prior to, during, and/or after the degradable component partially or fully degrades.

In another non-limiting object of the present invention is the provision of a degradable component wherein the tracer element is incorporated uniformly throughout the degradable component.

In another non-limiting object of the present invention is the provision of a degradable component wherein the tracer element is located on and/or within one or more specific areas or regions of the degradable component.

In another non-limiting object of the present invention is the provision of a degradable component wherein the degradable component includes one or more tracer elements in an amount of at least about 0.01 grams.

In another non-limiting object of the present invention is the provision of a degradable component wherein the degradable component includes one or more tracer elements in an amount of 0.01-10 grams (and all values and ranges therebetween).

In another non-limiting object of the present invention is the provision of a degradable component wherein the tracer element is in the form of an RFID tag, magnetic wire, or other information carrying device that is incorporated on and/or in a degradable component which is detectable using analytical techniques prior to, during, and/or after the degradable component partially or fully degrades.

In another non-limiting object of the present invention is the provision of a degradable component wherein the tracer element is in the form of a tracer molecule or element that is incorporated on and/or in a degradable component which is detectable using analytical techniques prior to, during, and/or after the degradable component partially or fully degrades.

In another non-limiting object of the present invention is the provision of a degradable component wherein the degradable component includes a plurality of tracer elements and wherein the plurality of tracer elements is the same.

In another non-limiting object of the present invention is the provision of a degradable component wherein the degradable component includes a plurality of tracer elements, and wherein some of the tracer elements are different from some of the other tracer elements.

In another non-limiting object of the present invention is the provision of a method for determining 1) whether a degradable component has begun to degrade, 2) the degree to which the degradable component has degraded, 3) whether the one or more particular regions of the degradable component has begun to degrade and/or the degree to which such one or more particular regions have degraded, and/or 4) whether the degradable component has been sufficiently removed from a location, wherein such method comprises the steps of a) providing a degradable component, b) providing one or more tracer elements (and if a plurality of tracer elements the tracer elements can be the same or different) that are incorporated on and/or in the degradable component, wherein the one or more tracer elements are configured to be released from the degradable component when the degradable component partially or fully degrades, c) exposing the degradable component to fluid which causes the degradable component to partially or fully degrade and thereby partially or fully releasing one or more tracers element from the degradable component that was previously located on and/or in the degradable component, and d) providing a detection arrangement (e.g., sensor, testing lab, visual inspection, etc.) to detect the presence and/or concentration of one or more tracer elements prior to release from the degradable component and/or after released from the degradable component due to the partial or full degradation of the degradable component.

In another non-limiting object of the present invention is the provision of a degradable component wherein 1) fluid can be analyzed by a sensor to detect the presence of one or more tracer elements, 2) samples of the fluid can be taken and tested by a sensor, some other testing device, or in a lab to test and/or detect the presence of one or more tracer elements, and/or 3) fluid can be visually detected to thereby detect the presence of one or more tracer elements.

In another non-limiting object of the present invention is the provision of a method of detection of the presence of a tracer element and/or the amount of detected tracer element to determine that 1) the degradable component has not degraded, 2) the degradable component has not sufficiently degraded, 3) a certain portion of the degradable component has not degraded, 4) a certain portion of the degradable component has begun to degrade, 5) the degree of degradation of one or more portions of the degradable component, and/or 6) the degradable component has sufficiently degraded.

In another non-limiting object of the present invention is the provision of a method used to determine the degradation status of a plurality of degradable components that are the same or different. Different tracer elements can be used to differentiate the degree of degradation of different components and/or different regions of components.

In another non-limiting object of the present invention is the provision of a method wherein the composition of the fluid and/or the flowrate of the fluid to which the degradable component is exposed can be used to 1) control a rate of degradation of the degradable component, 2) measure or estimate dissolution rates of the degradable component, 3) measure or estimate the degree of degradation of the degradable component, and/or 4) measure or estimate flow rates of fluids through specific fluids or zones in a well due to the progression of dissolution or degradation of the degradable component.

In another non-limiting object of the present invention is the provision of a method of using tracer elements that are the same or different from the one or more tracer elements in the degradable component that are inserted into the fluid so as to 1) determine if the fluid has encountered one or more of the degradable components, 2) determine the flow rate of the fluid about the one or more of the degradable component, and/or the flow of fluid through one or more regions of a well.

In another non-limiting object of the present invention is the provision of a method to assure that a degradable component has properly degraded by discretely locating the tracer element on and/or in one or more locations of the degradable component, detecting the presence of the tracer element, and/or by estimating the total amount of the tracer element released from the degradable component.

In another non-limiting object of the present invention is the provision of a tracer element that can be added into a pocket or cavity that has been machined into a tool or degradable component in an amount such that when the tool or degradable component partially or fully degrades, the tracer elements generates a readily detectable signal or is present in a concentration in the flowback or produced water that can be readily detected.

In another non-limiting object of the present invention is the provision of a tracer element that can be in a tool or degradable component in an amount such that when the tool or degradable component partially or fully degrades, the tracer elements generates a readily detectable signal or is present in a concentration in the flowback or produced water that can be readily detected.

In another non-limiting object of the present invention is the provision of tracer elements, such as chemical tracer elements, molecular compound tracer elements, elemental tracer elements, or isotope tracer elements, are present in an amount in and/or on the tool or degradable component so as to be detectable above the detection thresholds in the flowback water during the initial flowback, and/or later during produced water (e.g., water that flows through the well). Flowback normally occurs as part of the process of putting the well into production, generally from one day and three weeks after completing the well. Chemical tracers normally are detectable at sub-PPM to PPB levels, using available detection technologies. Radioisotopes generally have lower detection thresholds than salts or molecular tracers. The target level of tracer elements, such as chemical tracer elements, molecular compound tracer elements, elemental tracer elements, or isotope tracer elements in the flowback or produced water is about 0.01-10 ppm in the expected volumetric flow of flowback water and/or later during produced water.

In another non-limiting object of the present invention is the provision of tracer elements, such as chemical tracer elements, molecular compound tracer elements, elemental tracer elements, or isotope tracer elements are present in an amount in and/or on the tool or degradable component of about 5-500 grams (and all values and ranges therebetween) in a tool or degradable component, depending on expected water volume and flow duration during the dissolution and tracer release process.

In another non-limiting object of the present invention is the provision of tracer elements that is present in an amount in and/or on the tool or degradable component of at least about 0.01 wt. % of the tool or degradable component and less than 50 wt. % of the tool or degradable (and all values and ranges therebetween). In one non-limiting embodiment, the tracer element in the form of chemical tracer elements, molecular compound tracer elements, elemental tracer elements, and/or isotope tracer elements is generally present in an amount in and/or on the tool or degradable component of about 0.01-45 wt. % (and all values and ranges therebetween) of the tool or degradable component, and typically about 0.05-40 wt. % of the tool or degradable component.

Other objects, advantages, and novel features of the present invention will become apparent from the following detailed description of the invention when considered in conjunction with the accompanying drawings.

### BRIEF DESCRIPTION OF THE DRAWINGS

Reference may now be made to the drawings which illustrate various non-limiting embodiments that the invention may take in physical form and in certain parts and arrangement of parts wherein:

FIG. 1 illustrates a body formed of a degradable matrix that includes a plurality of chemical additives, and wherein the body is represented in three states: 1) the degradable matrix of the body has not dissolved or degraded; 2) the degradable matrix of the body has begun to degrade, but no chemical additive has been released from the body; and 3) the degradable matrix of the body has degraded to a point wherein chemical additive has been released from the body.

FIG. 2 illustrates a body formed of a degradable material that includes a cavity filled with a plurality of chemical additives, and wherein a degradable plug is used to retain the chemical additives in the cavity until the degradable material and/or the degradable plug has sufficiently degraded.

FIG. 3 illustrates a body formed of a degradable material wherein the chemical additives are uniformly dispersed throughout the degradable material.

FIG. 4 illustrates a body formed of a degradable material wherein the chemical additives are concentrated in a particular region of the degradable material, namely the center of the degradable material.

FIG. 5 illustrates a body formed of a degradable matrix that includes a plurality of tracer elements in the form of oxide particles, and wherein the body is represented in three states: 1) the degradable matrix of the body has not dissolved or degraded; 2) the degradable matrix of the body has begun to degrade, but no tracer element has been released from the body; and 3) the degradable matrix of the body has degraded to a point wherein tracer element has been released from the body.

FIG. 6 illustrates a body formed of a degradable material that includes a cavity filled with a plurality of tracer elements in the form of tracer tags, particles, and/or compound, and wherein a degradable plug is used to retain the tracer element in the cavity until the degradable material and/or the degradable plug has sufficiently degraded.

FIG. 7 illustrates a body formed of a degradable material wherein the tracer elements are uniformly dispersed throughout the degradable material.

FIG. 8 illustrates a body formed of a degradable material wherein the tracer elements are concentrated in a particular region of the degradable material, namely the center of the degradable material.

FIG. 9 illustrates two arrangements to control the timing of the release of the chemical additive from the degradable component.

FIG. 10 illustrates another arrangement to control the timing of the release of the chemical additive from the degradable component.

FIG. 11 illustrates another arrangement to control the timing of the release of two chemical additives from the degradable component.

FIG. 12 is a graph that illustrates the dissolution rates of magnesium alloy in the presence of various types of acids.

FIG. 13 is a table that illustrates the dissolution rates of magnesium alloy in the presence of various types of acids as represented in the graph of FIG. 12.

FIG. 14 illustrates a degradable metal ball that has been cut in half and cavities have been formed in each half for receiving a chemical additive in each half.

FIG. 15 illustrates a degradable ball that is exposed to different periods of time to a brine solution.

FIG. 16 illustrates the degradable ball of FIG. 15 after further exposure the ball to a brine solution.

### DESCRIPTION OF NON-LIMITING EMBODIMENTS

The present invention also relates to the enhanced use of degradable or dissolving tools and devices used in subterranean operations such as drilling, completion, and stimulation operations used in enhanced geothermal, oil and gas, and waste disposal (injection) operations wherein the degradable components include one or more chemical additives (e.g., salt, buffer chemical mixture, solid acid, or other active chemical) that are released upon the partial or full dissolution of the degradable component, and which the one or more chemical additives are used to at least partially control the local chemical environment about the degradable component to a) maintain the rate of degradation of the degradable component, b) enhance or accelerate the degradation of the degradable component, c) delay or slow the rate of degradation of the degradable component, and/or d) offset, neutralize or remove the byproducts of the degradation of the degradable component. The use of the one or more chemical additives can be used to ensure that the degradable component has sufficiently or completely degraded by ensuring that the environment about the degradable components is proper for the full or partial dissolution of the degradable component. Such proper environment can be fully or partially achieved by the inclusion of one or more chemical additives that are 1) coated on the degradable component, 2) incorporated in the composition of the degradable component, and/or 3) contained in one or more cavities of the degradable component. In one non-limiting aspect of the present invention, the one or more chemical additives can be released from the degradable component as ions/atoms, molecules, and/or particles species. The one or more chemical additives are generally salts, acids, and/or buffer materials such as, but not limited to, alkali or alkaline metals, bicarbonates, surfactants, etc. For example, the one or more chemical additives can be an enteric-coated solid acid or buffer particle that is used to neutralize a high pH solution, releasing acid only when the pH increases beyond a certain level. Such chemical additives can be used to limit or prevent Mg(OH)₂ build-up and to maintain degradation rates of the degradable component if poor fluid circulation occurs about the degradable component. The one or more chemical additives can optionally have controlled release properties by one or more mechanisms such as a degradable or dissolvable coating about the outer surface of the chemical additive, the particle size of the chemical additive, and/or the shape of the chemical additive. The one or more chemical additives can optionally be added in a desired amount and/or concentration into holes, or other features in the degradable component, and can be optionally covered, coated, plugged or sealed in or on the degradable component by a coating, a seal, or and/or adhesive. Such optional covering, coating, plug or seal can be used to control the timing of release of the one or more chemical additives from the degradable component and/or to limit or prevent removal of the one or more chemical additives from the degradable component during handling, shipment, and placement of the degradable component in the wellbore.

### Oil/Water Soluble Mesostructure Degradable Component

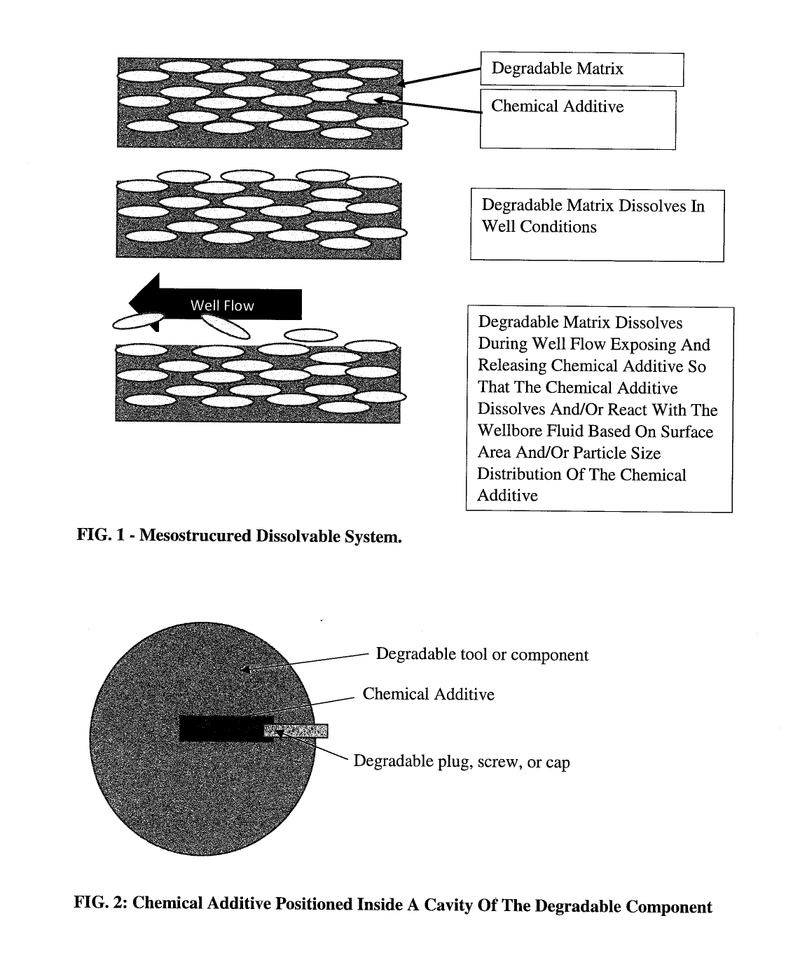

In one non-limiting embodiment of the invention, there is provided a mesostrucured chemical/degradable system where the degradable component is soluble in the wellbore (aqueous) fluid, there enabling the one or more chemical additives located on and/or in the degradable component to be exposed to the well flow conditions (See FIG. 1). Non-limiting chemical additives include KCL, NaCl, or other salt, solid acids such as sodium bisulfate, oxidizers or chlorine compounds such as perchlorates, or buffering acid such as oxalic acid. These chemical additives are configured to be released prior to and/or during the time that the degradable component degrades. One or more portions of the degradable component can be formed of a polymeric material; however, this is not required. As can be appreciated, the degradable component can be partially or fully made of other materials such as, but not limited to, a slightly soluble material or a controlled molecular weight compound. The one or more chemical additives that are released in the wellbore fluid dissolve in the wellbore fluid based on their particle size and/or particle size distribution, and/or through reaction with water to form an intermediate that creates a desired effect in the wellbore fluid.

The controlled release of the one or more chemical additives from the degradable component can be achieved by 1) adding a plaque or coating of material on the degradable component wherein the plaque or coating of material is formed of or includes the one or more chemical additives, 2) mechanically or adhesively attaching an extension, appendage, or additional section or component onto the degradable component wherein the attached component is formed of or includes the one or more chemical additives, and/or 3) including the one or more chemical additives or a material that includes the one or more chemical additives into recesses or cavities in the degradable component.

In one non-limiting embodiment of the invention, there is provided a controlled release chemical additive composite comprising a water-soluble chemical and a bio-degradable polymer and chemical additive. The chemical additive can be absorbed or dissolved into the water-soluble chemical and a bio-degradable polymer. Such water-soluble chemical and bio-degradable polymer can include an inorganic or organic material, such as a hydrogel material or absorbent material. Alternatively, the chemical additive can be in the form of a solid such as an acid or salt. In one non-limiting configuration, the bio-degradable polymer is a poly(α-hydroxyacid), such as poly(lactic acid), poly(glycolic acid), or blends thereof, a poly(orthoester), or a poly(anhydride), or a poly(hydroxyl alkanoate). The solid or composite material can be in the form of a particle. The particle can be a microparticle or a nanoparticle. The composite can alternatively be in the form of a gel, bulk scaffold, thin film or pellet, wherein the gel, bulk scaffold, thin film or pellet can optionally comprise particles of the chemical additive or composite that includes the chemical additive.

The composite that includes the chemical additive may further comprise an amphoteric species which is used to 1) alter the rate of dissolution of the inorganic material that forms the base of the composite material, 2) alter the rate of dissolution of the chemical additive in the composite material, 3) alter the rate at which the chemical additive is liberated from the composite material, and/or 4) function as a diffusion barrier to the chemical additive is liberated from the composite material.

The chemical additive and/or composite material that includes the chemical additive can be connected or otherwise secured to the degradable component by a binder. One non-limiting binder can be a water-soluble cellulose ether. Non-limiting examples of water-soluble cellulose ether binder is methylcellulose and/or hydroxypropylmethylcellulose.

The water-soluble cellulose ether binder can optionally be compounded with the chemical additive and a surfactant. Non-limiting surfactants which can be used include alkali metal sulfates of linear and branched alcohols, ethoxylated alcohols, ethoxylated alkylphenols, ethoxylated acids, ethoxylated amides, oils, fatty esters, alkali metal salts of sulfonates of naphthalene, alkylnaphthalenes, naphthalene condensates, alkyl-substituted benzenes, diphenyl derivatives, α-olefins, petroleum, oils, fatty acids, as well as the alkali metal salts of dialkyl sulfosuccinates, sodium or potassium dodecyl sulfate, sodium octadecyl sulfate, sodium sulfated castor oil, sodium dodecylbenzene sulfonate, sodium linear alkylate sulfonate, sodium sulfonated mineral oil, sodium petroleum sulfonate, sodium salt of naphthalenesulfonic acid-formaldehyde condensate and dioctyl sodium sulfosuccinate. The weight ratio of the surfactant to the water-soluble cellulose ether in the binder is generally 0.005-3:1, and the binder can include 5-95 wt. % of the water-soluble cellulose ether. The water-soluble cellulose ether can be used with or without prior humidification or similar treatment and when mixed with the surfactant and the chemical additive.

The degradable component can be partially for fully formed of a biodegradable polymer. The biodegradable polymer can be formed of a single polymer or be formed of a blend of at least two polymers. When the biodegradable polymer is formed of a blend of at least two polymers, the polymers can differ in their hydrophobicity, and/or be varied in concentration; however, this is not required.

### Localized Chemical Additive Incorporation

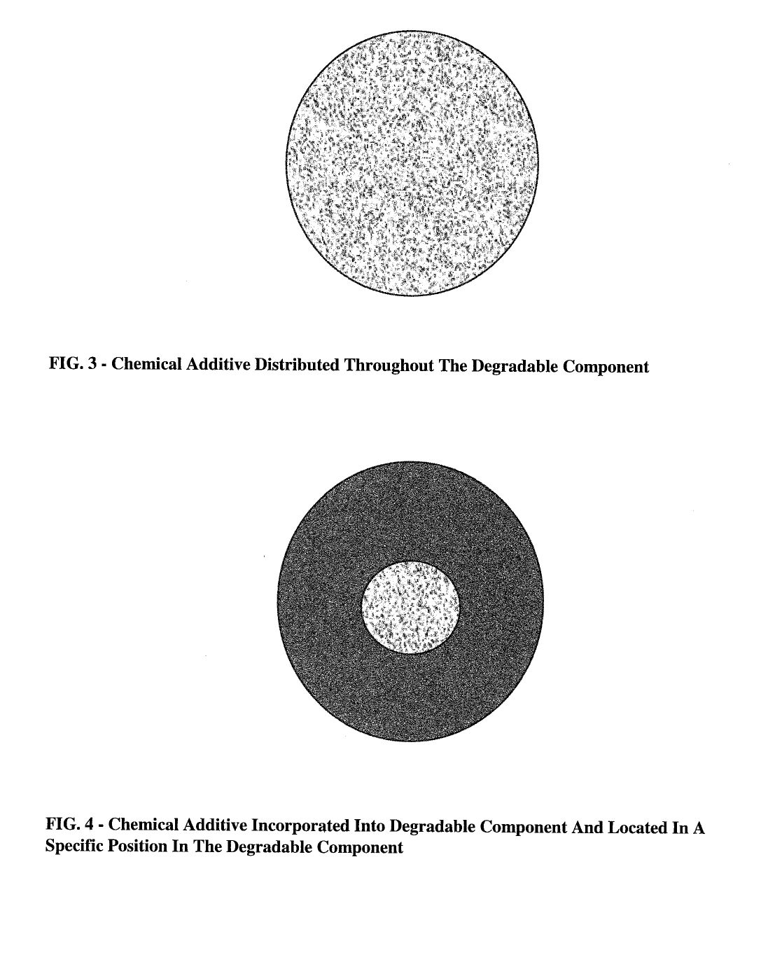

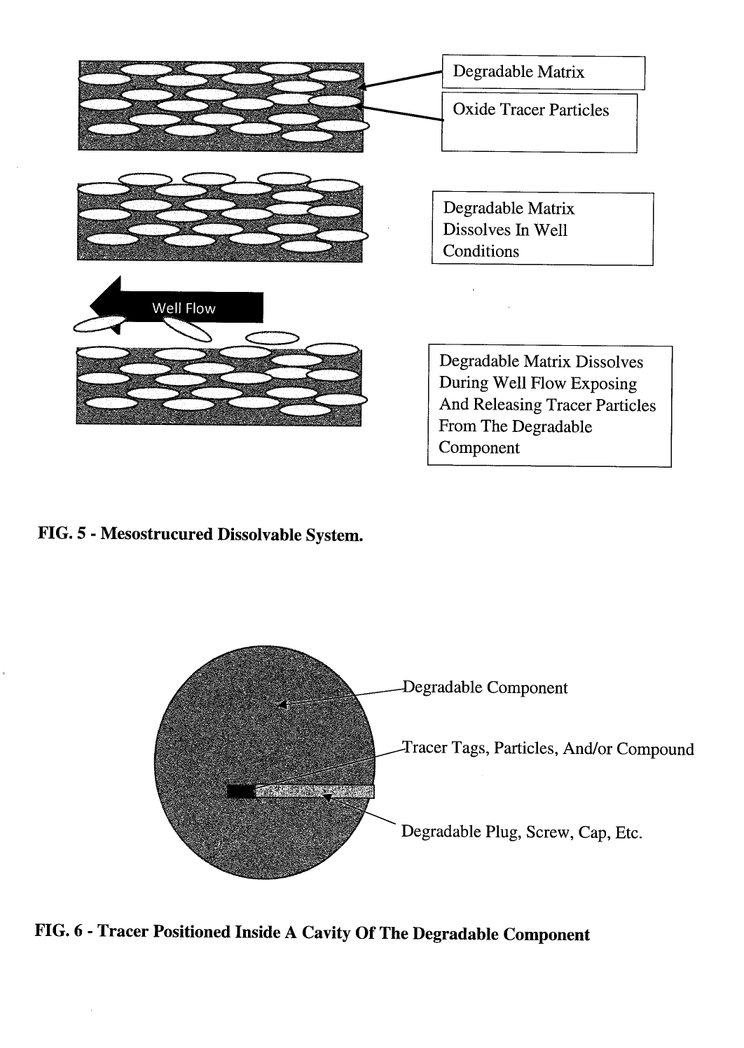

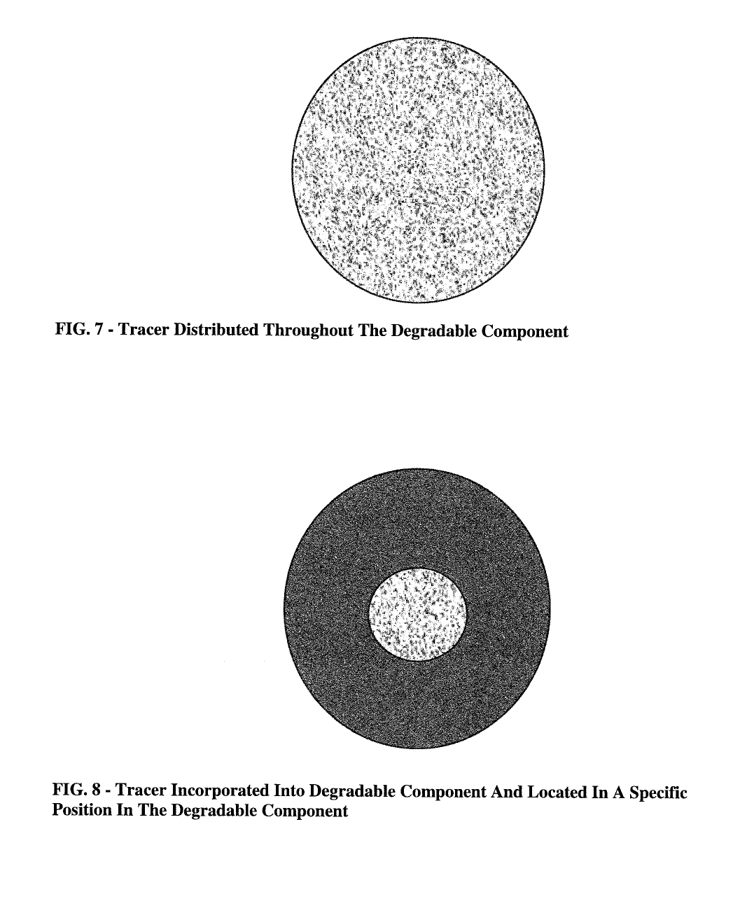

Referring to FIG. 1, the mesostrucured dissolvable system releases chemical additive continuously during degradation/dissolution of the degradable component. In some cases, a faster release rate of the chemical additive may be desired at a certain time that is prior to and/or during the degradation of the degradable component. As illustrated in FIG. 2, one or more chemical additives can be added at a select depth (such as the center of a frac ball, etc.), in a more concentrated or uninhibited (fast-release) form. On one non-limiting arrangement, a drilled hole can be formed in the degradable component, and the chemical additive can then be inserted into the drilled hole. The hole can then be optionally plugged with a degradable or permanent plug. In such an arrangement, the chemical additive in the hole is exposed to the wellbore fluid at a controlled time once the plug and/or has degraded a sufficient amount to expose the chemical additive to the wellbore fluid. An active agent such as an alkali metal or water-rechemical additive may be added in the hole to cause rapid mixing of the chemical additive in the wellbore fluid and/or rapid expulsion of the chemical additive from the hole due to gas creation or thermal energy production by the active agent so as to accelerate the chemical additive release from the degradable component and/or to accelerate reaction rate of the chemical additive with the wellbore fluid and/or degradable component once the cavity is exposed to the wellbore fluid. By adding controlled amounts of chemical additive to the degradable component that are exposed at selected or different times during the degradation of the degradable component, the dissolution rate of the degradable component can be accelerated, inhibited, or held constant, and the local environment, such as pH and salinity, can be controlled. FIGS. 3-4 illustrates other non-limiting arrangements for using a chemical additive with a degradable component in accordance with the present invention.

The use of chemical additives with the degradable component in accordance with the present invention can be used with components such as frac balls, bridge plugs, perforators, sleeves, liners, pintles, seals, etc. to ensure that such components are properly removed from a wellbore. The chemical additive can provide a controlled release of chloride (salt), oxidizer (chlorine), or acid to accelerate dissolution of the degradable component, and/or to offset high pH created by dissolution of metals, or low salinity in freshwater completions. The chemical additive can be useful in the release of acid or salt (Cl—) to 1) accelerate dissolution of the degradable component after the degradable component has completed its use, 2) reduce the probability of large debris being present in the wellbore that can cause damage to the completion or production strings during flowback, 3) allow for higher flowrates to be used during flowback and initial production, and/or 4) allow flowback to be initiated sooner than it could be without the chemical additions to the degradable component. Of particular use is the addition of chemical additives in the form of acids or salts to frac balls and frac plugs made of dissolvable metals. A significant amount of salt or acid chemical additive can be added to the degradable component. Such degradable component can optionally include a thin covering or coating to prevent the preliminary release of the chemical additive from the degradable component. Release of the chemical additive from the degradable component after an initial delay time can be used to cause rapid degradation of the remaining degradable component, thereby speeding dissolution of the degradable component and reducing time to production in the wellbore.

The present invention also relates to the enhanced use of degradable or dissolving tools and devices used in subterranean operations such as drilling, completion, and stimulation operations used in enhanced geothermal, oil and gas, and waste disposal (injection) operations wherein the degradable components include tracer elements that are released upon the partial or full dissolution of the degradable component, and which can be detected at the surface to ensure the desired degradation or removal of the degradable component.

In accordance with another and/or alternative aspect of the present invention, chemical tracers can be added into a pocket or cavity that has been machined into a tool and/or the chemical tracer can be added as a constituent or additive to the degradable material formulation of the tool to generate a readily detectable signal or concentration in the flowback or produced water. Tracer chemicals or isotopes need to be detectable above the detection thresholds in the flowback water during the initial flowback, or later during produced water for a slower dissolving tool or tool design to release tracers during production. Flowback normally occurs as part of the process of putting the well into production, generally from one day to three weeks after completing the well. Chemical tracers normally are detectable at sub-PPM to PPB levels, using available detection technologies. Radioisotopes generally have lower detection thresholds than salts or molecular tracers. The design and size of the cavity in the tool (or concentration in the tool for rare earth or radioisotopes incorporated into the alloy) is selected to result in a target level of chemical tracer in the flowback or produced water of from about 0.01-10 ppm in the expected volumetric flow during the time of dissolution or flow being analyzed. To obtain such concentrations in the flowback or produced water, the concentration of the tracer chemical in the tool is generally about 5-500 grams in a tool, depending on expected water volume and flow duration during the dissolution and tracer release process. Generally, the degradable component contains at least 1 wt. % tracer chemical. In one non-limiting specific embodiment, the degradable component contains about 1-45 wt. % tracer chemical (and all values and ranges therebetween).

### Example 1—Oil/Water Soluble Mesostructured Degradable Tracer

A mesostrucured tracer/degradable system is provided wherein the degradable component is soluble in the well (aqueous) fluid, thereby exposing the tracer element to the well flow conditions. The tracer element includes stable isotopes not common in the well formation, and which the tracer element is normally in the form of oxide or intermetallic particles. The tracer element is released from the degradable component as the degradable component degrades. The degradable component can be formed of a polymeric and/or metallic material. The released tracer elements can be analyzed on-site by testing the fluid flow or back flow of fluid from the well or by sending a sample of the fluid containing the tracer elements to an outside lab, typically a lab that uses high resolution GC-MS techniques; however, this is not required.

For example, the tracer element can be formed from rare earth oxide nanoparticles (CeO, Ge₂O₃, Sm₂O₃, Nd₂O₃, etc.) that are readily prepared using sol-gel synthesis and incorporated into the degradable component (e.g., polymeric degradable components, metallic degradable components, etc.). By sampling the flow or flowback water during completion, or at the start of well production, and partially evaporating the water, sensitivities in the ppt range can be achieved to detect the tracer elements in the tested fluid. The concentration of the tracer elements in the fluid is directly related to the volume of degradable component that has degraded when knowing the total flowback and a loss correction factor.

In the case of soluble degradable components such as polymerics, the tracer elements (e.g., oxide tracer particles, etc.) are released in proportion to the flow rate of the well section, and as the tracer elements are released, more polymer of the degradable component will be exposed to be dissolved in the well. By adding tracer elements to the degradable component such that they constitute a large percentage of the surface of the degradable component on dissolution, flow sensitivity (e.g., flow rates, etc.) of the fluid in the well can be increased by the detection of the tracer elements.

The detection of the tracer element can provide instantaneous degradation rates (how fast the degradation of the degradable component) as well as cumulative degradation of the degradable component (either sampling from total flowback, or averaged over total flowback, such as a sample from each container of flowback fluid). By adding different tracer elements to different zones or locations of the degradable component, the degradation of each zone of the degradable component can be determined. In one non-limiting example, the degradable component is a frac ball having a diameter of 1-5 inches. The tracer elements can be uniformly dispersed throughout the frac ball or be localized in the frac ball (e.g. inserted into a cavity in the frac ball). The frac ball can be formed of a metal or plastic material. The tracer element constituted less than 30 wt. % of the frac ball.

### Example 2—Localized Tracer Incorporation

The system as set forth in Example 1 releases tracer elements continuously during degradation/dissolution of the degradable component, and requires knowledge of the total flow of fluid past the degradable component and a recovery of a known percentage of the tracer element to assess the complete or desired amount of removal of the degradable component in the well. Such information can be facilitated by adding the tracer element at a select depth in the degradable component (such as the center of a degradable component [e.g., frac ball, etc.]). In such an arrangement, the tracer element can be added to the degradable component in a more concentrated form; however, this is not required if the release is detectable at lower concentrations. The insertion of the tracer element can be incorporated in the interior of the degradable component by various processes, for example, by drilling a hole or machining a cavity into the degradable component, placing the tracer element in the bottom of the cavity, and then plugging the hole. In this manner, any detection of the tracer element confirms full or sufficient removal of the degradable component. An alternate approach is to place the tracer in a pocket between, or at the intersection of, two components; for example, below the element or seal and the mandrel in a dissolvable frac or bridge plug such that when the tracer element is detected, it is known that the degradable component has been sufficiently degraded. The tracer element constituted less than 25 wt. % of the frac ball, and typically less than 10 wt. %. For example, the tracer element can constitute about 1-25 g.

### Example 3

A dissolvable metal frac ball having outer dimensions of 3.750 inches+/−0.003 inches is machined to form a hollow core having dimensions of 0.75 inch×2.5 inch. The hollow core thus constitutes less than 15 vol. % of the frac ball. The upper 1 inch of the cavity is machined with female NPT threads to accept a plug. One or more microRFID tags (e.g., 1-5 microRFID tags) having dimensions to fit into the hollow core (e.g., 0.6″ dia×0.1″) are coated with a coating to protect the one or more microRFID tags and/or provide buoyancy to the one or more microRFID tags so that the microRFID tags can float in the flowing water after being released form the degradable component. The coating is typically a non-degradable coating in the well fluid. The coating thickness is generally at least about 0.005 inches and typically about 0.01 inches to 0.1 inches (and all values and ranges therebetween). One non-limiting coating is a polyurethane coating. The coating can optionally can include about 0.1-70 vol. % (and all values and ranges therebetween) additive (e.g., microballoons, hollow spheres, high buoyance materials, etc.) to increase the buoyancy of the coating. One non-limiting additive are glass microballoons. In one non-limiting example, the microRFID tag can be coated with 0.02-0.05 inches of polyurethane which optionally contains about 30-35 vol. % glass microballoons. A dissolvable metal plug with matching male NPT threads is threaded into the cavity to form a seal to seal the cavity, then surface machined to the frac ball spherical surface to meet the frac ball diameter specifications. The frac ball is used during a normal stimulation process, and allowed to dissolve over 1 to 10 day period (e.g., 3-5 day period). A screening device is placed in the discharge of the flowback pipe, or a solids catcher is used in the flowback line to collect solids, typically greater than ⅛ inch or ¼ inch. The screening device is selected and designed so as to capture the microRFID tags. The screen, filter, or solids catcher are checked periodically or at the end of flowback as the well is close to connect to the production equipment. The information on the RFID tags is read by a portable reader to positively confirm complete dissolution of the frac ball, and to collect any other information the RFID tag or microcircuit has been constructed to collect. The tracer element constituted less than 25 wt. % of the frac ball, and typically less than 10 wt. %.

### Example 4

A dissolvable metal frac ball having outer dimensions of 3.750 inches+/−0.003 inches is machined to form a hollow core having dimensions of 0.75 inch×2.5 inch. The upper 1 inch of the cavity is machined with female NPT threads to accept a plug. 10-20 grams of a chemical tracer, such as, but not limited to, FFI 2300 from Spectrum Tracer Services, is then placed into the cavity. A dissolvable metal plug with matching male NPT threads is threaded into the cavity to form a seal, then surface machined to the ball spherical surface to meet the frac ball diameter specifications. The frac ball is used during a normal stimulation process, and allowed to dissolve over a 3-5 day period. Samples of the flowback water are collected periodically during completion, and sent to an analysis lab (in this case, Spectrum Tracer Services, LLC) for identification. Different tracers can be loaded into a series of frac balls (Spectrum Tracer Services, LLC has 41 FFI tracers available) to confirm that each stage of the well has completed dissolution of the frac balls in a particular section of the well. Identification of tracer elements from the toe stages confirmed that the well was open and flowing from all stages. The tracer element constituted less than 25 wt. % of the frac ball, and typically less than 10 wt. %.

In addition to chemicals detectable by analytical techniques (e.g., stable isotopes, high sensitivity molecules), microtags detectible using RF or other electromagnetic techniques can alternatively or additionally be used as the tracer element. One non-limiting example is to use a set of micro-RFID tags placed in the degradable component. The micto-RFID tags can be the only tracer elements or be used with other types of tracer elements (e.g. chemical tracers, etc.). A sufficient number of tags should be placed in the degradable component to ensure highly reliable detection in the flowback water or out-flowing water. These tracer elements can be detected in real time by flowing the produced/flowback fluid over or through a detection device. MicroRFID tags in the 100-300 micron range can be used and can be detected in a fluid flow using current detection technology. Medium or high frequency tags can be used, generally requiring recovery (such as by catching in a screen) during flowback and analysis, or low frequency tags, which are larger, have greater distance response particularly in water, and can more easily be analyzed on-line through the use of an antenna covering all or a portion of the flowback stream, with or without recovery of the tag. The tag can be engineered used to collect additional information, such as temperatures, salinity, pH, or other conditions occurring during the dissolution and exposure, and report those to the surface.

By adding unique tracer elements to different degradable components, the degradation and/or degradation rate of different degradable components can be independently monitored in the same flowback water. Also, by adding unique tracer elements to different regions of a degradable component, the degradation and/or degradation rate of a particular region or zone of degradable component can be independently monitored in the same flowback water.

The described invention is most commonly used to assure removal of components such as frac balls, bridge plugs, perforators, sleeves, liners, pintles, seals, etc. The method and system of the present invention also or alternatively can be used to detect and identify flows from the formation, such as by flowing produced fluid through a device including a degradable component, after which detection of the tracer element can provide information on water flows and rates (by concentration versus total flow). The adding of different types and/or compositions of tracer elements to different degradable components that are located in different zones of a well allows total water flow to be identified from each zone in the well. Such information can be used to control production and intervention activities in the well.

The degradable component can include one or more the one or more chemical additives such as a salt, solid acid, base, active chemical, or mixture (such as a eutectic salt mixture) to ensure the proper degradation or dissolution of the degradable material that partially or fully forms the degradable component. Of particular use are the addition of acids or salts to frac balls and frac plugs made of dissolvable metals. A significant amount of salt or acid can be added to the degradable component, with optionally a thin covering or coating (e.g., PVA, PGA, PLA, PEG, cellulose, sugar, poly(α-hydroxyacid) [e.g., poly(lactic acid), poly(glycolic acid], poly(orthoester), poly(anhydride), poly(hydroxyl alkanoate), gelatin, chitosan, arabinogalactan, collagen, alginate, hyaluronic acid, fibrin, cellulose ether, and/or other degradable polymer) to prevent preliminary release of the salt or acid form the degradable component. Release of the acid or salt after an initial delay of time causes rapid degradation of the remaining degradable materials of the degradable component, thereby speeding dissolution of the degradable component and reducing time to production in a wellbore.

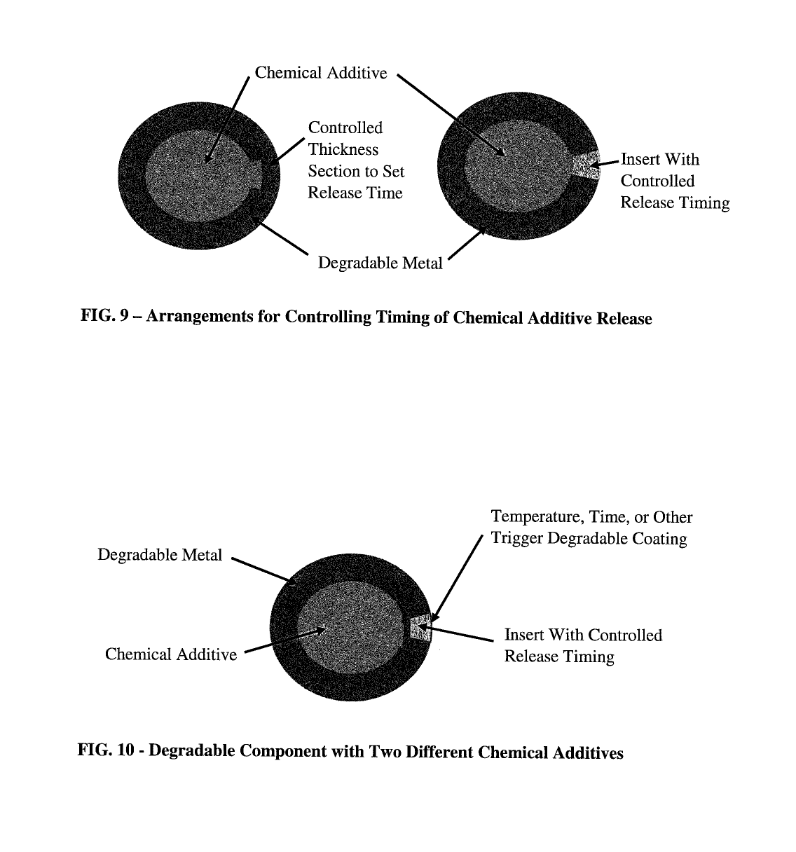

Referring now to FIGS. 9-11, there is illustrated four different arrangements for controlling the timing of the release of the chemical additive from a cavity degradable component. The balls (e.g., frac balls, etc.) illustrated in FIGS. 9-11 include a cavity that includes one or more different types of chemical additives (e.g., acid, salt, buffer agent, etc.). Non-limiting chemical additives include salts of KCl, NaCl, CaCl₂, NaBr, KBr, MgCl₂, AlCl₂, AlBr₃, BF₃, AlF₃, KI, NaI, ZnCl₂, ZnBr₂, CuCl₃, acids of carboxylic acids (steric acid, benzoic acid, maleic acid, malonic acid, etc.), solid acids such as phosphoric acid, sulfates such as sodium sulfate, sulfur oxide, and acid chloride such as ethonyl chloride, benzoic chloride. In one non-limiting arrangement, the chemical additive is a solid material. The size, shape and number of cavities in the balls is non-limiting. The total volume of the one or more cavities in the degradable component generally constitutes at least 10% of the total volume of the degradable component, typically about 10-90% (and all values and ranges therebetween) of the total volume of the degradable component (e.g., balls, etc.). As illustrated in FIGS. 9-11, the material used to for the degradable balls is a degradable metal material; however, it can be appreciated that the material can alternatively be a degradable polymer. The amount and concentration of the one or more chemical additives in the cavity of the degradable component is present such that such the one or more chemical additives in the cavity of the degradable component can cause at least 30 vol. % (e.g., 30-100%) of the material used to for the degradable balls to degrade or dissolve.

The ball illustrated on the left side of FIG. 9 includes a generally spherical cavity (however other cavity shapes can be used) wherein one portion of the cavity wall is thinner. As such, the thickness of one or more portions of the cavity wall can be adjusted in thickness to control the timing of release of the one or more chemical additives from the cavity of the ball. The size and shape of the thinned wall portion can also be used to control the rate of release of the one or more chemical additives from the cavity once the thinned wall portion has sufficient degraded or dissolved to all for the release of the one or more chemical additives from the cavity.

The ball illustrated on the right side of FIG. 9 also includes a generally spherical cavity (however other cavity shapes can be used). An opening is formed in one or more portions of the cavity wall and a plug is illustrated as being press-fit, threaded or otherwise connected in the opening. The plug may be a different 1) composition from composition the composition of the material used to for the degradable balls, 2) different density from the material used to for the degradable balls, or 3) have some other difference that results in the degradation rate or dissolution rate of the plug to be different (e.g., greater) than the degradation rate or dissolution rate of the composition of the material used to for the degradable balls; however, this is not required. Generally, the plug is designed to degrade or dissolve to allow for release of the one or more chemical additives from the cavity prior to the time that the cavity wall of the ball has sufficiently degraded or dissolved to allow for the release of the one or more chemical additives from the cavity.

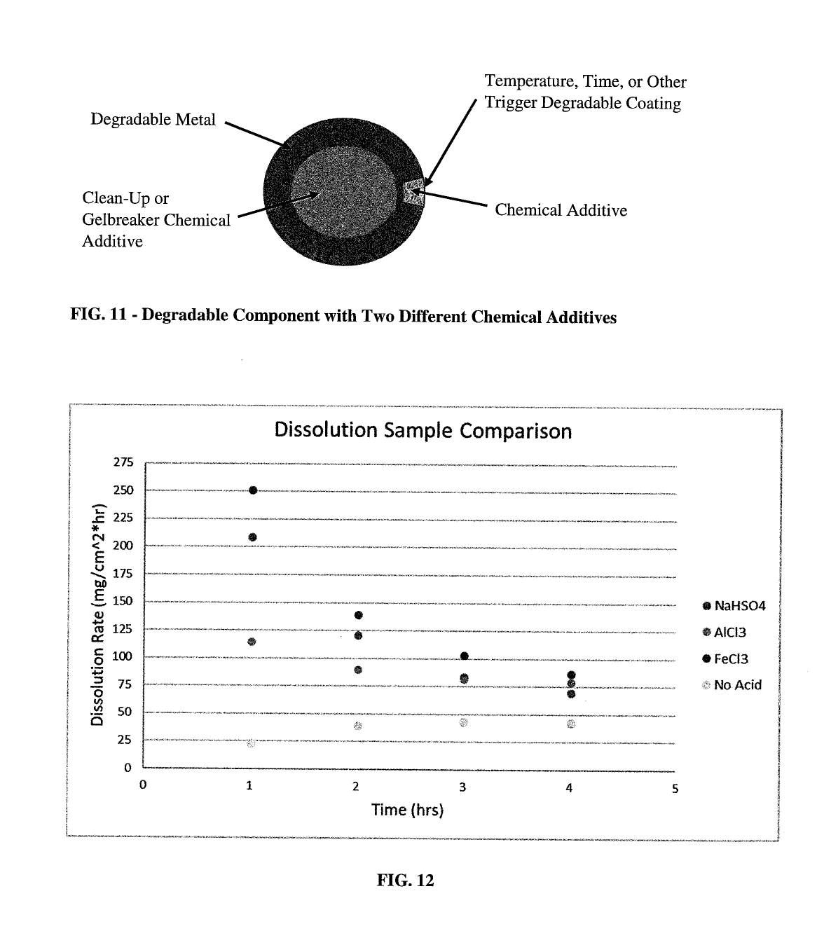

The ball illustrated in FIG. 10 includes both a thinned walled cavity portion and a plug positioned adjacent to the thinned walled cavity portion. In addition, the plug can optionally include a degradable coating material (e.g., PVA, PGA, PLA, PEG, cellulose, sugar, poly(α-hydroxyacid) [e.g., poly(lactic acid), poly(glycolic acid], poly(orthoester), poly(anhydride), poly(hydroxyl alkanoate), gelatin, chitosan, arabinogalactan, collagen, alginate, hyaluronic acid, fibrin, cellulose ether, and/or other degradable polymer) to control the time period at which the plug begins to degrade or dissolve. The trigger that causes the optional degradable coating on the plug to sufficiently dissolve or degrade such that the plug material is exposed to fluid that causes the plug to begin to dissolve or degrade can be a temperature trigger, pH trigger, pressure trigger, fluid composition trigger, etc. The thickness and/or composition of the coating can also or alternatively be used to provide the desired time delay before the plug sufficiently dissolves or degrades such that the plug material is exposed to fluid that causes the plug to begin to dissolve or degrade. As can be appreciated, a degradable coating (not shown) can be optionally coated on one or more portions or all of the outer surface of the ball to delay the time period at which the degradable material (e.g., degradable metal) of is exposed to fluid that causes the degradable material to begin to dissolve or degrade. Such a degradable coating could optionally be used on the balls illustrated in FIGS. 9 and 11. The composition of the degradable coating is generally different from the composition of the plug. Likewise, when the degradable coating is coated on to the outer surface of the degradable material of the degradable component, the composition of the degradable coating is generally different from the composition of the degradable material, and also generally has a thickness that is less than the cavity wall thickness.

As illustrated in FIG. 10, the thickness of the plug is greater than the thickness of the thinned portion of the cavity wall; however, this is not required. As such, this non-limiting embodiment of the ball combines the two control mechanism illustrated in FIG. 9 to control the release of the one or more chemical additives from the cavity of the ball.

Referring now to FIG. 11, the ball includes a thinned walled cavity portion and an indent or surface recession that container a slug of chemical additive slug. The slug is illustrated as being positioned adjacent to the thinned walled cavity portion. The slug of chemical additive can include one or more different chemical additives. In one non-limiting arrangement, the slug of chemical additive includes two or more types of chemical additives (e.g., KCl, NaCl, etc.). Generally, the composition of the slug of chemical additive is different from the composition of the chemical additive in the cavity of the ball; however, this is not required. In one non-limiting arrangement, the slug of chemical additive includes KCl and/or NaCl, and the chemical additive in the cavity includes a gelbreaker chemical (e.g., NaHSO₄, etc.). Generally, the volume amount of chemical additive in the slug is less than the volume amount chemical additive in the cavity of the ball.

The slug of chemical additive can optionally include a degradable coating material (e.g., PVA, PGA, PLA, PEG, cellulose, sugar, poly(α-hydroxyacid) [e.g., poly(lactic acid), poly(glycolic acid], poly(orthoester), poly(anhydride), poly(hydroxyl alkanoate), gelatin, chitosan, arabinogalactan, collagen, alginate, hyaluronic acid, fibrin, cellulose ether, and/or other degradable polymer) to control the time period at which the slug begins to degrade or dissolve. The trigger that causes the optional degradable coating on the slug to sufficiently dissolve or degrade such that the plug material is exposed to fluid that causes the slug to begin to dissolve or degrade can be a temperature trigger, pH trigger, pressure trigger, fluid composition trigger, etc. The thickness and/or composition of the coating can also or alternatively be used to provide the desired time delay before the slug sufficiently dissolves or degrades such that the plug material is exposed to fluid that causes the slug to begin to dissolve or degrade. As can be appreciated, the degradable coating can be used with or replaced by a degradable cap (e.g., plug, etc.) or other type of degradable cover for the slug.

In one non-limiting operation, the slug of chemical additive is designed to be released prior to the release of the chemical additive in the cavity of the ball. The slug of chemical additive is generally selected to have a composition that facilitates in the degradation or dissolution of the degradable material of the ball. After the ball has been sufficiently degraded, the chemical additive in the cavity of the ball is released. The composition of the chemical additive in the cavity of the ball can be selected to facilitate in providing self clean-up of degradable metal byproducts by causing them to become soluble in the wellbore fluid. As can be appreciated, the chemical additive in the cavity of the ball can also or alternatively be used to facilitate in the degradation or dissolution of the degradable material of the ball.

The chemical additive located in the cavity of the ball and/or the slug of chemical additive as illustrated in FIGS. 9-11 can be melt processable so as to maximize the amount of solid acid and/or salt added to the degradable component. In one non-limiting embodiment, the acid and/or salt is melt processable wherein it can be poured into a mold and then 1) be directly placed in a cavity and/or recess of a degradable component, 2) be coated with a degradable coating and then be directly placed in a cavity and/or recess of a degradable component, and/or 3) be cast directly into the degradable component.

As can be appreciated, the cast solid chemical additive and/or the cast solid chemical additive that is coated with a degradable coating can be attached or otherwise secured on or connected adjacent to the degradable component (e.g., a plug, mandrel, shoe, barrier, disc, dart, or other component or device). In one non-limiting embodiment, the chemical additive includes a solid acid such as, but not limited to, FeCl₃, AlCl₃, and/or Na₂SO₄. The ratio of the solid acid to the degradable metal can be selected such as to shift the degradation byproducts and/or solution pH away from insoluble hydroxides to soluble sulfates or chorides or oxychlorides. The following chemical equations illustrate the approximate stoichiometric ratios for the complete reaction of different salts and mangesium.

Reaction 1

| | | | |
|---|---|---|---|
| 2 FeCl₃ + | 1 H₂O → | 1 Fe₂O₃ + | 6 HCl |
| 162.204 g/mol. | 18.02 g/mol. | 156.69 g/mol. | 36.46 g/mol. |
| 324.408 g | 18.02 g | 156.69 g | 218.76 g |

Reaction 2

| | | | |
|---|---|---|---|
| 1 Mg + | 2 HCl → | 1 MgCl₂ + | 1 H₂ |
| 24.305 g/mol. | 36.46 g/mol. | 95.211 g/mol. | 2 g/mol. |
| 24.305 g | 72.92 g | 95.211 g | 2 g |

Reaction 3

| | | | |
|---|---|---|---|
| 1 AlCl₃ + | 3 H₂O → | 1 Al(OH)₃ + | 3 HCl |
| 133.34 g/mol. | 18.02 g/mol. | 77.98 g/mol. | 36.46 g/mol. |
| 133.34 g | 56.06 g | 77.98 g | 109.38 g |

Reaction 4

| | | | |
|---|---|---|---|
| 1NHSO₄ + | 1 H₂O → | 1 NaOH + | 1 H₂SO₄ |
| 120.06 g/mol. | 18.02 g/mol. | 39.99 g/mol. | 98.079 g/mol. |
| 120.06 g | 18.02 g | 39.99 g | 98.079 g |

Reaction 5

| | | | |
|---|---|---|---|
| 1 Mg + | 1 H₂SO₄ → | 1 MgSO₄ + | 1 H₂ |
| 24.305 g/mol. | 98.079 g/mol. | 120.365 g/mol. | 2 g/mol. |
| 24.305 g | 98.079 g | 120.365 g | 2 g |

Based on the above reactions, 47.56 g (0.2932 mol.) of FeCl₃ will make about 0.8796 mol. of HCl which can consume about 10.69 g (0.4398 mol.) of Mg.

Also based on the above reactions, 40.672 g (0.305 mol.) of AlCl₃ will make about 0.915 mol. of HCl which can consume about 11.12 g (0.457 mol.) of Mg.

Also based on the above reactions, 44.97 g (0.375 mol.) of NHSO₄ will make about 0.375 H₂SO which can consume about 9.1 g (0.374 mol.) of Mg.

Generally, the degradable component includes sufficient chemical additive to enable a solubilization (reaction) of at least 35% of the degradable material of the degradable component and, generally, sufficient chemical additive to enable a solubilization (reaction) of at least 80% of the degradable material of the degradable component. In some non-limiting embodiments, the degradable component includes sufficient chemical additive to enable a solubilization (reaction) of over 100% of the degradable material of the degradable component. As such, in some arrangements, to ensure complete removal of the degradable material of the degradable component, a stoichiometric amount of chemical additive to degradable material is used in the degradable component. It has been found that to inhibit or prevent the possibility of plugging and/or cementing of sand grains, the degradable component includes sufficient chemical additive to enable a solubilization (reaction) of 50-150% of the degradable material of the degradable component and, typically the degradable component includes sufficient chemical additive to enable a solubilization (reaction) of 80-120% of the degradable material of the degradable component.

Examples of stoichiometric amounts for magnesium for certain reactions are:

4.5 g FeCl₃ per gram of Mg, or 2.76 cc FeCl₃ per cc of Mg (MgOH+⅔ FeCl₃+H₂O→MgCl₂+Fe(OH)₃.

3.7 g AlCl₃ per gram of Mg, or 1.35 cc of AlCl₃ per cc of Mg

4.94 g NaHSO₄ per gram of Mg or 3.24 cc NaHSO₄ per cc of Mg.

Although a larger volume of NaHSO₄ is required to dissolve Mg than compared to HCl, NaHSO₄ is generally less hazardous to the wellbore materials than the HCl that is generated from the solid acid chlorides. Also NaHSO₄ is a low cost material and is easier to handle than FeCl₃ and AlCl₃ salts.

A mixture of salts can be added to the degradable component to 1) accelerate dissolution of the degradable material of the degradable component, 2) reduce or eliminate the sensitivity of the degradable component to wellbore fluid salinities, and/or 3) enable dissolvable metals in the degradable component to be dissolvable in freshwater wells. One non-limiting salt mixture that can be used is KCl/NaCl.

In another non-limiting embodiment, a plurality of different chemical additives can be included in the degradable component. In one non-limiting arrangement, a plurality of different chemical additives are located on different regions of the degradable component. For example, an chemical additive in the form of one or more solid acids can be located in an interior cavity of the degradable component, and one or more salts can be located closer to and/or on the surface of the degradable component, and wherein the one or more solid acids and one or more salts are spaced from one another and do not contact one another on the degradable component. In such an arrangement, the one or more solid acids and one or more salts may interact with the fluid about the degradable component at differing times. One such arrangement is illustrated in FIG. 11. Such an arrangement can be used to both eliminate sensitivity of the degradable component to the chloride content about the degradable component, while also creating soluble byproducts and conditions to prevent the need for subsequent intervention and wellbore clean-up.

In another non-limiting embodiment, the chemical additive on the degradable component can be principally used for the purpose of wellbore cleanup. For example, the degradable component can include one or more solid acids having controlled release from the degradable component for use in removing cements and/or filter cakes in the wellbore, and/or to deliver wellbore cleanup or gelbreaking chemistries to the wellbore. In such an embodiment, the amount of chemical additive can be included in the degradable component, or be separately added to a brine or other type of well fluid. When the chemical additive is to be used principally for purposes of wellbore cleanup, the amount of the chemical additive can maximized, such as by forming a solid shape of the solid acid or other type of chemical additive, and then 1) optionally coating the solid chemical additive with a degradable coating, and/or 2) placing the solid chemical additive inside a degradable shell of the degradable component. When the chemical additive is to be added directly to a well fluid, the chemical additive typically includes a degradable coating (e.g., PVA, PGA, PLA, PEG, cellulose, sugar, poly(α-hydroxyacid) [e.g., poly(lactic acid), poly(glycolic acid], poly(orthoester), poly(anhydride), poly(hydroxyl alkanoate), gelatin, chitosan, arabinogalactan, collagen, alginate, hyaluronic acid, fibrin, cellulose ether, and/or other degradable polymer). PVA coatings have been found to be particularly effective in providing a temperature-controlled release of the chemical additive in a wellbore. When precise location of release of the chemical additive is needed in the wellbore, a degradable rubber wedge or wiper can be added to the degradable-encapsulated chemical additive to produce a pump-down dart, plug, or device that inhibits or prevents fluid leakage around the component or device until the chemical additive is positioned in a desired location in the wellbore. Alternately or additionally, the component or device that includes the chemical additive can accommodate and/or be attached to a slickline or wireline so as to precisely locate the component or device in the wellbore for release of the chemical additive in a desired position in the wellbore.

### Example 5

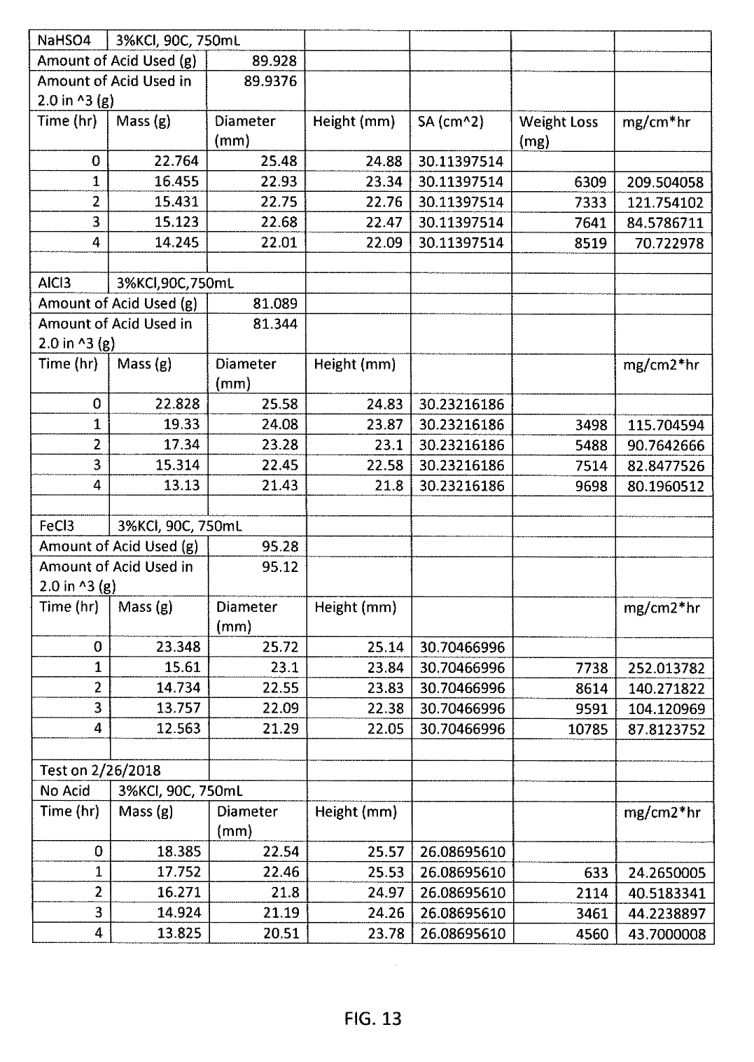

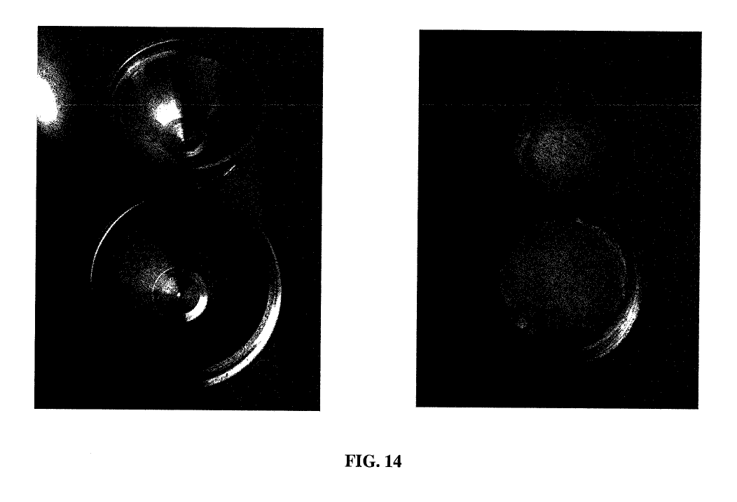

Solid acids were added to TervAlloy™ TAx100 buttons (degradable magnesium alloy buttons) by placing a solid acid in a beaker that contained the buttons. FIGS. 12-13 illustrate a table and graph showing the dissolution rate over time of the magnesium alloy button in the presence of different chemical additives.

### Example 6

A 3.75″ TAx100 degradable magnesium frac ball was machined in two halves with a wall thickness of 0.250″, and NaHSO₄ was poured into each of the two halves as illustrated in FIG. 13. Thereafter, a metal grade epoxy was used to bond the halves together. The frac ball releases acid from the NaHSO₄ in the cavity of the frac ball between 6 and 14 hours after being inserted into a wellbore having 3 vol. % KCl fluid at a temperature of 70-110° C., and the frac ball completely dissolved in under 36 hours in the wellbore.

### Example 7

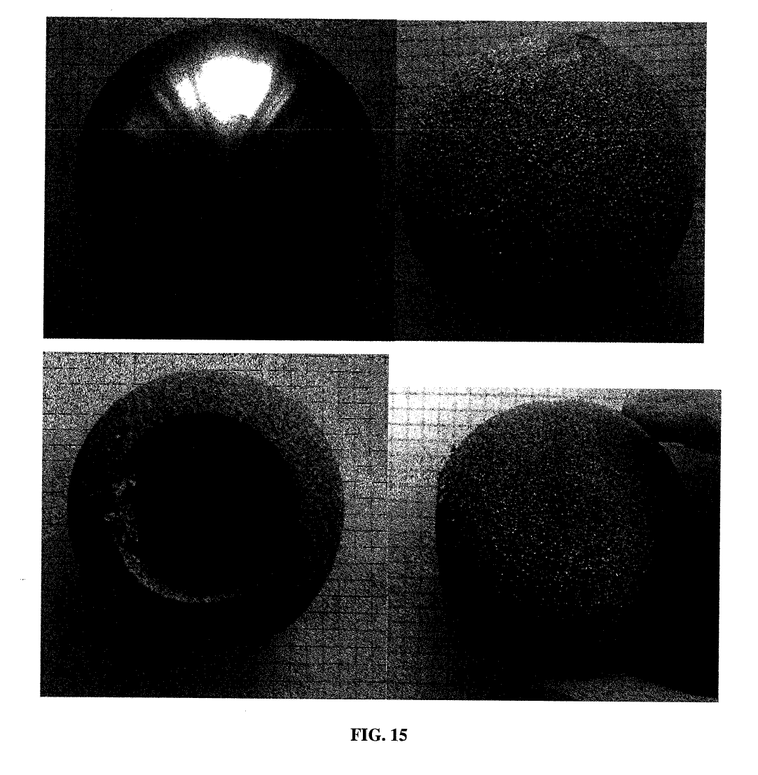

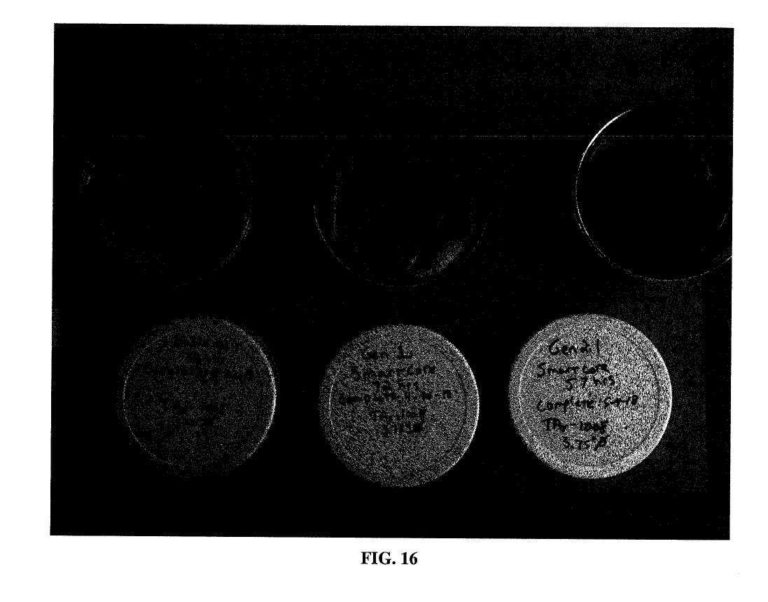

A 3.75″ degradable magnesium ball was machined to form a cavity in the ball that had a maximum wall thickness of 0.1455″. A core access point was then formed in the ball. After machining of the ball was complete, NaHSO₄ is poured into the core of the ball via the core access point to fill the entire volume of the cavity. The cavity was thereafter sealed using a tapered plug that was inserted into the core access point. The ball release the acid from NaHSO₄ between 6 and 14 hours after being placed into a wellbore with 3 vol. % NaCl fluid at a temperature of 70-110° C. FIG. 15 illustrates the ball prior to be exposed to a solution of 3 vol. % NaCl fluid at a temperature of 70-110° C. (top-left figure), and the ball after being exposed to 4-24 hours in a solution of 3 vol. % NaCl fluid at a temperature of 70-110° C. The ball is illustrated after exposure of 4-24 hours to the NaCl fluid to have partially degraded, but the plug that retains the NaHSO₄ in the cavity of the ball was still intact and little or no NaHSO₄ was released from the ball during such time period. FIG. 16 illustrates the ball after 57 and 72 hours in the NaCl fluid. The ball has completely dissolved within 60 hours. The ball was observed to have been dissolved about 85% within 36 hours. As such, after the plug degraded to a point wherein the NaHSO₄ in the cavity was released from the ball, the ball experienced rapid degradation or dissolution.

### Example 8

A cylindrical container is fabricated from a degradable structural material such as TAx100 degradable magnesium alloy. The structural container is cylindrical, with a 6″ diameter and 3/32″ wall thickness. A hemispherical cap was threaded onto the top end of the container. The container has a total length of 24″. 18 lbs. of AlCl₃ was melted and then poured into the cavity of the degradable container. The rear end the container was thereafter sealed with a degradable cap. A degradable elastomer wiper ring is attached to a groove in the outer surface of the container to create a seals between the container and a 7″ production liner. The container was pumped down into the wellbore to a specific location in the wellbore, wherein the container interacted with the aqueous wellbore fluid. After a period of time in the wellbore (5-60 hours) in a 3 vol. % KCl fluid at a temperature of 70-110° C., the container and plug degraded to allow for the release of the AlCl₃ from the cavity of the container. The AlCl₃ in the container was sufficient to clean-up and acidify a 25-50 ft. section of the wellbore, or to completely remove 5 lbs. of magnesium from such wellbore section by causing the magnesium to dissolve.

### Example 9

Multiple degradable cylindrical containers as described in Example 8 were placed in a wellbore. A first degradable cylinder was added to the wellbore. Sufficient wellbore fluid (approximately 100 gallons) was added behind the container to create a 50 ft. fluid column Another container is then added, and this process is repeated for every 50 ft. of fluid until a desired treatment length was staged in the wellbore. The staged length was then pumped down to a predetermined depth into the wellbore by pumping the requisite amount of fluid (approximately 2 gallons/ft. for a 7″ ID liner. The AlCl₃ in the cavity of the containers was sufficient to remove 3/32″ of CaCO₃ filter cake from the treated region of the wellbore.

It will thus be seen that the objects set forth above, among those made apparent from the preceding description, are efficiently attained, and since certain changes may be made in the constructions set forth without departing from the spirit and scope of the invention, it is intended that all matter contained in the above description and shown in the accompanying drawings shall be interpreted as illustrative and not in a limiting sense. The invention has been described with reference to preferred and alternate embodiments. Modifications and alterations will become apparent to those skilled in the art upon reading and understanding the detailed discussion of the invention provided herein. This invention is intended to include all such modifications and alterations insofar as they come within the scope of the present invention. It is also to be understood that the following claims are intended to cover all of the generic and specific features of the invention herein described and all statements of the scope of the invention, which, as a matter of language, might be said to fall there between. The invention has been described with reference to the preferred embodiments. These and other modifications of the preferred embodiments as well as other embodiments of the invention will be obvious from the disclosure herein, whereby the foregoing descriptive matter is to be interpreted merely as illustrative of the invention and not as a limitation. It is intended to include all such modifications and alterations insofar as they come within the scope of the appended claims.

What is claimed:

1. A method of using a degradable component comprising:
a. providing a degradable component that at least partially forms a tool or device;
b. providing a chemical additive or a matrix material that includes the chemical additive on, in, or attached to the degradable component, wherein the chemical additive is formulated to influence the degradation of the tool;
c. placing the degradable component in a wellbore;
d. providing a wellbore fluid in the area around the degradable component; and,
e. releasing the chemical additive in a controlled manner into the local environment by interaction with wellbore fluid.

2. The method as defined in claim 1, wherein the chemical additive is selected from an acid, buffer compound, salt, oxidizer, water rechemical additive, surfactant, and/or absorbent material.

3. The method as defined in claim 1, wherein the chemical additive is in the form of particles with a particle size distribution, where the solubility or reaction rate of the chemical additive can be controlled by the particle size and/or particle size distribution.

4. The method as defined in claim 1, wherein the chemical additive is incorporated into a compound or matrix that has a solubility or degradability in the wellbore fluid such as to control release of the chemical additive in the wellbore fluid.

5. The method as defined in claim 1, wherein the degradable component is at least partially formed of said matrix material, and the matrix material is selected from a water-soluble or water-reactive polymer or compound.

6. The method as defined in claim 1, wherein the chemical additive is at least partially incorporated in said matrix material, and the matrix material is selected from a water-soluble or water-reactive polymer or compound.

7. The method as defined in claim 1, wherein said matrix material includes one or more components selected from the group consisting of PVA, PGA, PEG, sugar, cellulose, a poly(α-hydroxyacid) such as poly(lactic acid), poly(glycolic acid), or blends thereof, a poly(orthoester), a poly(anhydride), a poly(hydroxyl alkanoate), gelatin, chitosan, arabinogalactan, collagen, alginate, hyaluronic acid, fibrin, cellulose, and cellulose ether.

8. The method as defined in claim 1, wherein the chemical additive includes one or more components selected from the group consisting of a salt including KCl, NaCl, CaCl₂, NaBr, KBr, MgCl₂, AlCl₃, AlBr₃, BF₃, AlF₃, KI, NaI, ZnCl₂, ZnBr₂, CuCl₃, an acid including carboxylic acids (steric acid, benzoic acid, maleic acid, malonic acid, etc.), solid acids such as phosphoric acid, sulfates such as sodium sulfate, sulfur oxide, and acid chloride such as ethonyl chloride, benzoic chloride, etc.

9. The method as defined in claim 1, wherein the chemical additive or matrix that includes the chemical additive is compressed to form a solid pellet.

10. The method as defined in claim 1, wherein the chemical additive or matrix that includes the chemical additive is in the form of granules, pellets, or powders.

11. The method as defined in claim 1, wherein the chemical additive or matrix that includes the chemical additive is coated to control a release rate of the chemical additive into the wellbore fluid.

12. The method as defined in claim 1, wherein the chemical additive is added to a cavity in the degradable component.

13. The method as defined in claim 12, wherein the cavity is formed in a frac ball, or a component in a tool such as a bridge or frac plug.

14. The method as defined in claim 1, wherein the component is a mandrel, cone, element, or shoe.

15. The method as defined in claim 1, wherein the chemical additive or matrix material that includes the chemical additive is exposed at the surface of the degradable component.

16. The method as defined in claim 1, wherein the chemical additive or matrix material that includes the chemical additive is exposed by mechanical action, such as shear, sliding, pressure pulse, etc., or by dissolution of a coating or plug covering a cavity in the degradable component, wherein such plug can be the same or different material as the degradable component.

17. The method as defined in claim 1, wherein the chemical additive or matrix material that includes the chemical additive is added as an additional component to the degradable component, such as an extension to the shoe, mandrel extension, lining, or cylinder, and where such additional chemical component may be mechanically or adhesively attached to the degradable component.

18. The method as defined in claim 1, wherein the chemical additive is added as a coating or a lining to some or all of the degradable component or surface of the degradable component.

19. The method as defined in claim 1, wherein the chemical additive is added in an amount of 1-50 wt. % of the degradable component, and generally from 3-10 wt. % of the degradable component.

20. The method as defined in claim 1, wherein the chemical additive is formulated to partially or fully neutralize the formation of hydroxides and/or to maintain a pH about the degradable component of below 10, and generally below 8, and typically below 6.

21. The method as defined in claim 1, wherein the chemical additive is present on and/or in the degradable component to produce 1000-10000 ppm of chloride content in the wellbore fluid about the degradable component when the chemical additive is released in the wellbore fluid, and typically 3000-5000 ppm chloride content.

22. The method as defined in claim 1, wherein the chemical additive is protected within a recess of the degradable component, or protected by an extension or cover designed to prevent mechanical damage and/or control wellbore fluid access to the chemical additive.

23. The method as defined in claim 1, wherein the chemical additive includes a salt, solid acid, base, active chemical, mixture (such as a eutectic salt mixture) that can be melted and poured into a cavity of the degradable component.

24. The method as defined in claim 1, wherein the chemical additive is melted in its hydrate or water-containing form and thereafter is poured into a cavity of the degradable component and then heated to remove 90-100% of the water in the chemical additive so that the chemical additive solidifies in its anhydrous, or lower H₂O content form.

25. The method as defined in claim 1, wherein the pouring of the melted chemical additive into the cavity of the degradable component does not cause the metal to react or dissolve more than 10% while a) the chemical additive is in its molten state, and/or b) during the time of forming the degradable component with the chemical additive, and the chemical additive in the formed degradable component causes less than 10% degradation to the metal properties of the degradable component over a period of at least 1 month while the degradable component is stored in dry and ambient conditions.

26. The method as defined in claim 1, wherein the chemical additive can be mixed with another acid salt or compound that forms a eutectic mixture to allow that combined material to be melted and poured into a cavity of degradable component at a temperature that will not adversely affect the degradable component.

27. The method as defined in claim 1, wherein the addition of the chemical additive to the degradable component has no significant effect on the mechanical performance of the degradable component when the degradable component is placed in compression since the solid chemical additive is incompressible.

28. The method as defined in claim 1, wherein the amount or stoichiometric amount of the chemical additive on and/or in the degradable component is equal to or is more than is required to dissolve or to cause dissolution of at least 30% of the degradable material of the degradable component.

29. The method as defined in claim 28, wherein the amount or stoichiometric amount of the chemical additive on and/or in the degradable component is equal to or is more than is required to dissolve or to cause dissolution of over 100% of the degradable material of the degradable component.

30. The method as defined in claim 28, wherein the amount or stoichiometric amount of the chemical additive on and/or in the degradable component is equal to or is more than is required to dissolve or to cause dissolution of at least 30% of the degradable material of the degradable component, and the chemical additive is used to cause a) the dissolving or dissolution of the degradable material of the degradable component, and/or b) the dissolving or dissolution of other degradable materials in close proximity of the degradable component.

31. The method as defined in claim 28, wherein the amount or stoichiometric amount of the chemical additive on and/or in the degradable component is equal to or is more than is required to dissolve or to cause dissolution of 30-200% of the degradable material of the degradable component, and the chemical additive is used to cause a) the dissolving or dissolution of the degradable material of the degradable component, and/or b) the dissolving or dissolution of other degradable materials in close proximity of the degradable component.

32. The method as defined in claim 1, wherein the degradable component includes a dissolvable metal shell encompassing a solid chemical additive.

33. The method as defined in claim 32, wherein the degradable component includes a plug, bridge plug, frac plug, and/or thinned wall layer to close an opening into a cavity of the degradable component.

34. The method as defined in claim 33, wherein the degradable component includes a secondary chemical additive that is used to enhance the rate at which the plug, bridge plug, frac plug, and/or thinned wall portion of the cavity dissolves or degrades so that the chemical additive in the cavity of the degradable component is at least partially released from the cavity in less than 72 hours.

35. The method as defined in claim 33, wherein the chemical additive in the cavity of the degradable component is sealed in the cavity by a watertight threaded plug, interference fit plug, and/or polymer sealing compound, and where the sealing plug and/or polymer sealing compound can have the same or different degradation rate than the degradable material of the degradable component.

36. The method as defined in claim 33, wherein the degradation rate and/or thickness of a portion of the dissolvable plug, bridge plug, frac plug, and/or thinned wall portion of the cavity is designed to control the timing of the release of the chemical additive from the cavity.

37. The method as defined in claim 1, wherein the amount or stoichiometric amount of the chemical additive on and/or in the degradable component causes 70-100% the degradable component to degrade or dissolve in less than 72 hours.

38. The method as defined in claim 1, wherein the degradable component includes a cavity that has one or more chemical additives, and that the degradable component can withstand more than 5 ksi differential pressure on a seat or hydrostatic pressure of 5 ksi or more.

39. The method as defined in claim 1, wherein the chemical additive is a solid acid, acidic pH buffer or other active chemical that causes the byproducts of the degradable device when the degradable component dissolves to become more soluble in a fluid such as water.

40. The method as defined in claim 39, wherein the degradable material of the degradable component is magnesium alloy, zinc alloy, or aluminum alloy, or other degradable metal, and the byproducts of the degradable device when the degradable component dissolves include magnesium hydroxide, aluminum hydroxide, zinc hydroxide, and/or other metal hydroxide.

41. The method as defined in claim 1, wherein the degradable component is designed to deliver a chemical treatment to a specific wellbore location in a time-controlled manner.

42. The method as defined in claim 41, wherein said degradable component is delivered to a specific wellbore location by using a slickline, wireline, controlled orifice size, logging or active signal, pumping amount of liquid, or other technique.

43. The method as defined in claim 41, wherein said degradable component is in the form of a container that includes a cavity that contains one or more chemical additives, wherein said container is at least partially formed of a degradable metal or polymer.

44. The method as defined in claim 41, wherein said degradable component includes a degradable sealing ring such as a degradable plastic or elastomeric wiper or flap to create a seal with the wellbore when said degradable component is inserted into a wellbore.

45. The method as defined in claim 1, wherein said degradable component includes an amount of chemical additive to enable a predetermined amount of additive to be delivered to a location in the wellbore, wherein the amount of the chemical additive is sufficient to solubilize 70-130% of the hydroxide and/or carbonate that is present in the wellbore in the form of a filter cake and/or degradable metal byproduct.

46. A method of monitoring or confirming dissolution of a degradable component as defined in claim 1 that includes the steps of:
a. providing a degradable component that is partially or fully formed of a degradable material and includes one or more tracer elements;
b. placing said degradable component downhole into the bore or near-bore area of a well formation;
c. causing said degradable component to at least partially degrade or dissolve and to partially or fully release said one or more tracer elements from said degradable component; and,
d. recovering, collecting, monitoring or analyzing said one or more tracer elements to confirm dissolution or degradation of said degradable component or a degree of dissolution or degradation of said degradable component to thereby determine whether desired bore access has been obtained in said subterranean operation.

47. A degradable component that includes a) a body at least partially formed of a degradable metal and/or polymer and b) one or more chemical additives, said one or more chemical additives positioned i) on an outer surface of said body, b) incorporated in said body, c) positioned in one or more cavities in said body, d) positioned in one or more recesses in said body, e) positioned in a coating on said body, and/or f) positioned in a plug in said body, said one or more chemical additives formulated to cause said degradable metal and/or polymer to at least partially dissolve or degrade.

48. The degradable component as defined in claim 48, wherein the chemical additive includes one or more components selected from the group consisting of a salt including KCl, NaCl, CaCl₂, NaBr, KBr, MgCl₂, AlCl₃, AlBr₃, BF₃, AlF₃, KI, NaI, ZnCl₂, ZnBr₂, CuCl₃, an acid including carboxylic acids (steric acid, benzoic acid, maleic acid, malonic acid, etc.), solid acids such as phosphoric acid, sulfates such as sodium sulfate, sulfur oxide, and acid chloride such as ethonyl chloride, benzoic chloride, etc.

49. The degradable component as defined in claim 49, wherein said chemical additive is a solid salt, solid acid, solid base, and/or solid eutectic salt mixture.
